(19) United States
(12) Patent Application Publication
(10) Pub. No.: US 2002/0045220 A1
(43) Pub. Date: Apr. 18, 2002

(54) Biosynthesis of polyketide synthase substrates

(76) Inventors: Chaitan Khosla, Palo Alto, CA (US); Blaine Pfeifer, Stan Ford, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/798,033

(22) Filed: Feb. 28, 2001

Related U.S. Application Data
(63) Continuation-in-part of application No. 09/687,855, filed on Oct. 13, 2000.
(60) Provisional application No. 60/159,090, filed on Oct. 13, 1999.
(60) Provisional application No. 60/206,082, filed on May 18, 2000.
(60) Provisional application No. 60/232,379, filed on Sep. 14, 2000.

(51) Int. Cl. C12P 19/62; C07H 17/08; C12N 1/21

(52) U.S. Cl. 435/76; 435/252.3; 435/252.33; 536/7.1

(57) ABSTRACT

The use of enzymes which catalyze the production of starter and extender units for polyketides is described. In addition, modified loading modules are described, which can accept a variety of starting units such as substituted benzoates, and which can be used to generate substituted derivatives of natural products. These enzymes may be used to enhance the yield of polyketides that are natively produced or polyketides that are rationally designed. By using these techniques, the synthesis of a complete polyketide has been achieved in E. col. This achievement permits a host organism with desirable characteristics to be used in the production of such polyketides and to assess the results of gene shuffling.

### CROSS-REFERENCE TO RELATED APPLICATIONS

This application is related to application Ser. No. 60/159,090 filed Oct. 13, 1999; Ser. No. 60/206,082 filed May 18, 2000; and Ser. No. 60/232,379 filed Sep. 14, 2000, which are expressly incorporated herein by reference. This application is a continuation in part application of U.S. application Ser. No. 09/687,855 filed Oct. 13, 2000, which is relied on and incorporated herein by reference in its entirety.

### STATEMENT OF RIGHTS TO INVENTIONS MADE UNDER FEDERALLY SPONSORED RESEARCH

This invention was made with U.S. government support from the National Institutes of Health and the National Science Foundation. The U.S. government may have certain rights in this invention.

### TECHNICAL FIELD

The invention relates to methods to adapt procaryotic hosts for efficient production of polyketides. In one aspect, the hosts are modified to synthesize the starter and/or extender units used by polyketide synthases in the synthesis of polyketides. In another aspect, hosts are modified to synthesize synthases which accept substituted benzoates as starter units. Other host modifications may also be made. Thus, the invention includes methods for production of complex polyketides in such diverse organisms as Escherichia coli, Bacillus, Myxococcus, and Streptomyces.

### BACKGROUND ART

Rifamycin B

The rifamycin synthetase is primed with a 3-amino-5-hydroxybenzoate (AHB) starter unit by a loading module that contains domains homologous to the adenylation (A) and thiolation (T) domains of nonribosomal peptide synthetases. The rifamycin synthetase of Amycolatopsis mediterranei is responsible for the biosynthesis of prosansamycin X, a precursor to the antibiotic rifamycin B (FIG. 1). (The protein complex responsible for biosynthesis of prosansamycin X, a rifamycin B precursor, is referred to herein as rifamycin synthetase because the results described herein establish that ATP is required for covalent attachment of the aryl starter unit to the loading module of the complex.) The rifamycin synthetase consists of a core of five multifunctional proteins, RifA, RifB, RifC, RifD, and RifE, in addition to RifF, a protein that is believed to cyclize the linear product of the other proteins via intramolecular amide formation (Schupp, T., et al. (1998) FEMS Microbiol. Lett. 159, 201-207; August, P. R., et al. (1998) Chem. Biol. 5, 69-79; Tang, L., et al. (1998) Gene 216, 255-265; Floss, H. G., et al. (1999) Curr. Opin. Chem. Biol. 3, 592-597). The five multifunctional proteins can be further subdivided into one nonribosomal peptide synthetase (NRPS)-like loading module and ten polyketide synthase (PKS) modules, based on sequence homology to other systems.

RifA, the N-terminal protein component of rifamycin synthetase, contains an NRPS-like module, the adenylation-thiolation (A-T) loading didomain, upstream of the first condensing module (FIG. 1). The first such A-T type loading module was identified in the gene cluster for the natural product rapamycin (Schwecke, T., et al. (1995) Proc. Natl. Acad. Sci. USA 92, 7839-7843). Complete gene clusters for other synthetases that contain hybrid modular interfaces have since been reported (Gehring, A. M., et al. (1998) Chem. Biol. 5, 573-586; Quadri, L. E. N., et al. (1998) Chem. Biol. 5, 631-645; Silakowski, B., et al.(1999) J. Biol. Chem. 274, 37391-37399; Julien, B., et al. (2000) Gene 249, 153-160.; Tillett, D., et al. (2000) Chem. Biol. 7, 753-764; Wu, K., et al. (2000) Gene 251, 81-90; Du, L., et al. (2000) Chem. Biol. 7, 623-640), and these synthetases produce hybrid natural products that are composed of both ketide and peptide units. The proven track record of polyketide and peptide natural products as therapeutics suggests that the increased combinatorial diversity embodied in hybrid products will advance drug discovery. It would be advantageous to use a biochemical understanding of hybrid synthetases coupled with the ability to manipulate hybrid interfaces through protein engineering to enable the potential of such hybrid molecules to be realized.

The NRPS-like A-T didomain of RifA presumably primes the synthetase with 3-amino-5-hydroxybenzoate (AHB), which has been shown to be the precursor of the mC₇N structural element of rifamycin B (FIG. 1) (Ghisalba, O. et al. (1981) J. Antibiot. 34, 64-71; Anderson, M. G., et al. (1989) J. Chem. Soc. Chem. Commun., 311-313). However, the mechanism of this priming has not been established. Two alternative models can be envisioned. In the coenzyme A (CoA) ligase model prevalent in the literature (Schupp, T., et al. (1998) FEMS Microbiol. Lett. 159, 201-207; August, P. R., et al. (1998) Chem. Biol. 5, 69-79; Ghisalba, O. et al. (1981) J. Antibiot. 34, 64-71), the activated AHB-adenylate product of the A domain is attacked by CoA to generate an AHB-CoA intermediate, and the aryl thioester enzyme intermediate results from transthiolation onto the T domain (FIG. 2A). In an alternative mechanism, which has been confirmed as detailed below, that is analogous to the mechanism used to prime NRPS modules, AHB is activated as the aryl-adenylate by the A domain, and the thiol of the phosphopantetheine cofactor of the T domain attacks AHB-adenylate directly to form a covalent aryl thioester enzyme intermediate (FIG. 2B).

Although AHB is the natural substrate of the A-T didomain, previous in vivo studies have revealed that RifA can be primed by the alternative substrates 3-hydroxybenzoate (3-HB) and 3,5-dihydroxybenzoate (Hunziker, D., et al. (1998) J. Am. Chem. Soc. 120, 1092-1093). It would be advantageous to harness this innate substrate tolerance for its implications for the production of unnatural natural products. In one aspect, it would be advantageous to reconstitute the activity of the A-T didomain of rifamycin synthetase in vitro in order to establish the mechanism of this priming module and to systematically investigate its substrate tolerance. Thus, the invention provides homologous substituted substrates for the production of unnatural natural products.

6-Deoxyerythronolide B

Erythromycin, a broad spectrum antibiotic synthesized by the bacterium Saccharopolyspora erythraea, is a prototype of a class of complex natural products called polyketides (O'Hagan, D., The Polyketide Metabolites (Ellis Horwood, Chichester, U. K., 1991). Complex polyketides such as 6-deoxyerythronolide B (6-dEB), the macrocyclic core of the antibiotic erythromycin, constitute an important class of natural products. These biomolecules are synthesized from simple building blocks such as acetyl-CoA, propionyl-CoA, malonyl-CoA and methylmalonyl-CoA through the action of large modular megasynthases called polyketide synthases (Cane, D. E., et al., Science 282:63 (1998)), generally found in actinomycetes. For example, the polyketide synthase (PKS) which results in the synthesis of 6-dEB is produced in Sacromyces erythraea. The polyketides produced in these native hosts are generally subsequently tailored to obtain the finished antibiotic by glycosylation, oxidation, hydroxylation and other modifying reactions. Polyketide structural complexity often precludes the development of practical laboratory synthetic routes, leaving fermentation as the only viable source for the commercial production of these pharmaceutically and agriculturally useful agents. At the same time, the challenges associated with developing scalable and economically feasible fermentation processes for polyketide production from natural biological sources (principally the Actinomyces family of bacteria) are enormous, and represent the most serious bottleneck during polyketide pre-clinical and clinical development. Recent work from this laboratory has demonstrated that it is possible to express polyketide synthase modules in a functional form in Escherichia coli (Gokhale, R. S., et al., Science (1999) 284:482-485). However, in order to harness these modular enzymes for polyketide biosynthesis in E. coli, or in other hosts that do not normally produce them it is also necessary to produce their appropriate substrates in vivo in a controlled manner. For example, metabolites such as acetyl-CoA, propionyl-CoA, malonyl-CoA and methylmalonyl-CoA are the most common substrates of these enzymes. E. coli has the capability to produce acetyl-CoA, propionyl-CoA, and malonyl-CoA; however, the latter two substrates are only present in small quantities in the cell, and their biosynthesis is tightly controlled. The ability of E. coli to synthesize methylmalonyl-CoA has not been documented thus far.

Similar conditions prevail in other microbial cells, especially those that do not natively produce polyketides, such as various species of Escherichia, Bacillus, Pseudomonas, and Flavobacterium. Thus, generally, the required starter and/or extender units may not be produced in adequate amounts in any particular host. Further, by appropriate selection of the acyl transferase (AT) domains of the PKS in question, substrates more complex than those just mentioned may be employed. As an example, the PKS for synthesis of FK506 comprises an acyl transferase domain that incorporates substrates such as propyl malonyl-CoA in preference to malonyl-CoA or methylmalonyl-CoA. It would be helpful to have available a method which provides this range of substrates in appropriate levels in any arbitrarily chosen host organism.

Additional problems that may need to be surmounted in effecting the production of polyketides in procaryotic hosts, especially those which do not natively produce polyketides, include the presence of enzymes which catabolize the required starter and/or extender units, such as the enzymes encoded by the prp operon of E. coli, which are responsible for catabolism of exogenous propionate as a carbon and energy source in this organism. In order to optimize production of a polyketide which utilizes propionyl CoA as a starter unit and/or utilizes its carboxylation product, methylmalonyl CoA as an extender unit, this operon should be disabled, except for that portion (the E locus) which encodes a propionyl CoA synthetase. Any additional loci which encode catabolizing enzymes for starter or extender units are also advantageously disabled.

In addition, a particular procaryotic host, such as E. coli, may lack the phosphopantetheinyl transferase required for activation of the polyketide synthase. It may be required to modify the host to contain such a transferase as well.

In summary, it would be advantageous to effect the production of polyketides in microbial, especially procaryotic hosts in general, and, in particular, in hosts which do not natively produce polyketides. These hosts often have advantages over native polyketide producers such as Streptomyces in terms of ease of transformation, ability to grow rapidly in culture, and the like. These advantages are particularly useful in assessing the results of random mutagenesis or gene shuffling of polyketide synthases. Thus, the invention provides a multiplicity of approaches to adapt microbial hosts for the production of polyketides.

Disclosure of the Invention

The invention has also achieved, for the first time, the production of a complete polyketide product, 6-dEB, in the ubiquitously useful host organism, E. coli. The methods used to achieve this result are adaptable to microbial hosts in general, especially procaryotics. They can be used to adapt microbial hosts which do not natively produce polyketides to such production and to enhance the production of polyketides in hosts that normally produce them. Depending on the host chosen, the modifications required may include incorporation into the organism of expression systems for the polyketide synthase genes themselves; disabling of endogenous genes which encode catabolic enzymes for the starter and/or extender units; incorporation of expression systems for enzymes required for post translational modification of the synthases, such as phosphopantetheinyl transferase; and incorporation of enzymes which enhance the levels of starter and/or extender units. The particular combination of modifications required to adapt the host will vary with the nature of the polyketide desired and with the nature of the host itself.

Thus, in one aspect, the invention is directed to microbial host cells which are genetically modified for enhanced synthesis of at least one polyketide wherein said modification comprises incorporation of at least one expression system for producing a protein that catalyzes the production of starter and/or extender units and/or disabling at least one endogenous pathway for catabolism of starter and/or extender units.

In one aspect, the invention is directed to the production of a polyketide product comprising substituted benzoates used as starter unit substrates for a A-T loading didomain of a rifamycin synthetase to make modified polyketides in organisms such as E. coli. In another aspect, the invention includes a screening method to determine which substituted benzoate derivatives are viable substrates for an A-T didomain.

Additional modifications may also be made, such as incorporating at least one expression system for a polyketide synthase protein and, if necessary, incorporating at least one expression system for a phosphopantetheinyl transferase.

In other aspects, the invention is directed to methods of preparing polyketides, including complete polyketides, in the modified cells of the invention. A preferred embodiment is a method to synthesize 6-dEB, 6-dEB analogs or other complete polyketides in E. coli.

In still another aspect, the invention is directed to a method to assess the results of gene shuffling or random mutagenesis of polyketide synthase genes by taking advantage of the high transformation efficiency of E. coli. An assay for polyketide production is also contemplated.

### BRIEF DESCRIPTION OF THE DRAWINGS

FIG. 1 is a proposed biosynthetic scheme for prosansamycin X, a precursor to rifamycin B. The rifamycin synthetase consists of a core of five large multifunctional proteins, RifA, RifB, RifC, RifD, and RifE, each containing one or more PKS modules. Each PKS module catalyzes one cycle of chain extension and associated β-ketoreduction for the biosynthesis of prosansamycin X. The N-terminal A-T loading didomain of RifA primes the synthetase with AHB and is reminiscent of a minimal NRPS module. The location of the mC₇N unit derived from AHB is shown in bold in the prosansamycin X structure. The active sites denote adenylation (A), thiolation (T), acyltransferase (AT), ketosynthase (KS), β-ketoreductase (KR), or dehydratase (DH) domains. As indicated, RifF is believed to catalyze cyclization via intramolecular amide formation.

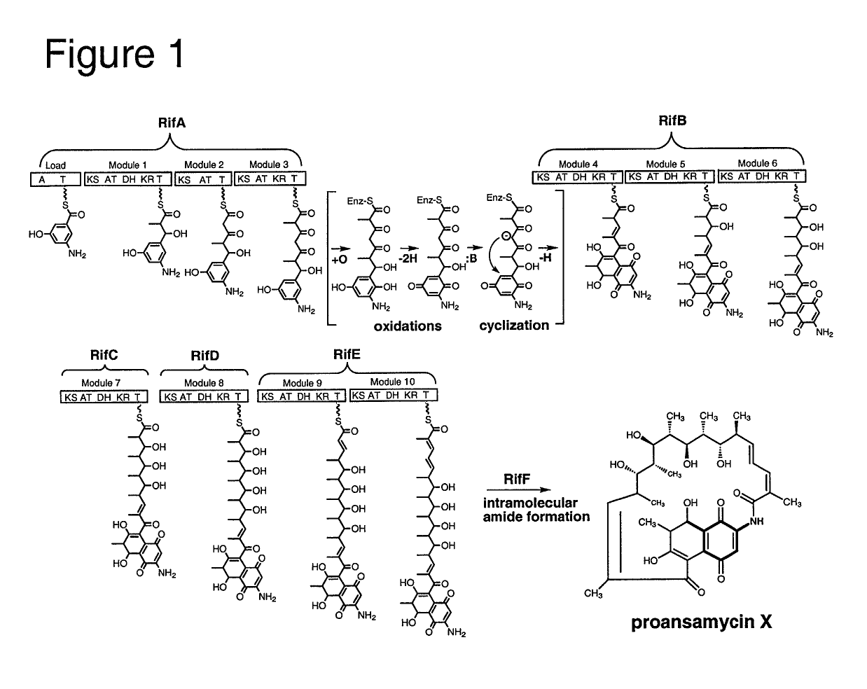

FIG. 2 illustrates possible mechanisms for the A-T loading didomain. (A) In the CoA ligase model, the activated AHB-adenylate product of the A domain is attacked by CoA to generate an AHB-CoA intermediate, and the aryl thioester enzyme intermediate results from transthiolation onto the T domain. (B) In the NRPS-like mechanism, AHB is activated as the AHB-adenylate by the A domain, and the thiol of the phosphopantetheine cofactor of the T domain attacks AHB-adenylate directly to form a covalent aryl thioester enzyme intermediate.

FIG. 3 is a chart showing the presence or absence of apo or holo A-T didomain, ATP, and [¹⁴C]-B or [¹⁴C]-3-HB based on the results of the ATP-dependent covalent loading of the holo A-T didomain with B and 3-HB is shown based on Coomassie-stained gel (4-15% gradient) of the reaction mixtures and an autoradiograph of this gel (not shown).

FIG. 4 graphs the high performance liquid chromatography (HPLC) traces of time courses of reactions containing the apo A-T didomain. No net formation of benzoyl-CoA is observed. Labeled peaks were identified by co-injection with authentic standards of CoA, B, and benzoyl-CoA. The HPLC traces were shifted progressively by 0.15 min.

FIG. 5 graphs the saturation curves for covalent loading of the holo A-T didomain by 3-HB (□) or B (O). FIG. 5A is a linear representation of the data. FIG. 5B is a logarithmic representation of the data to facilitate evaluation of both data sets simultaneously. The lines are best fits of the data to a simple saturation model and give k_{cat}=1.9 min⁻¹ and K_{M}=180 μM for 3-HB, and k_{cat}=0.14 min⁻¹ and K_{M}=170 μM for B.

FIG. 6 shows the two plasmids used a synthetic operon approach to facilitate the expression of the DEBS and PCC genes. The restriction sites are abbreviated as follows: X, XbaI; N, NdeI; E, EcoRI; H, HindIII; B, Bpul102I; Ns, NsiI; Ps, PstI; P, PacI; D, DraIII.

FIG. 7A is a schematic of the 6-deoxyerythronolide B synthase (DEBS). The catalytic domains are: KS, ketosynthase; AT, acyl transferase; ACP, acyl carrier protein; KR, ketoreductase; ER, enoyl reductase; DH, dehydratase, TE, thioesterase. DEBS utilizes 1 mole of propionyl-CoA and 6 moles of (2S)-methylmalonyl-CoA to synthesize 1 mole of 6-deoxyerythronolide B (6dEB, compound 1). FIG. 7B illustrates that truncated DEBS1+TE produces the triketide lactone (compound 2). FIG. 7C illustrates that the rifamycin synthetase is a polyketide synthase that is naturally primed by a nonribosomal peptide synthetase loading module, comprised of two domains- an ATP dependent adenylation domain (A) and a thiolation domain (T). Substitution of this A-T didomain in place of the loading didomain of DEBS yields an engineered “hybrid” synthase that utilizes exogenous acids such as benzoic acid to synthesize substituted macrocycles such as compound 3 in an engineered strain of E. coli.

FIG. 8 is a schematic of the genetic design of E. coli BAP 1.

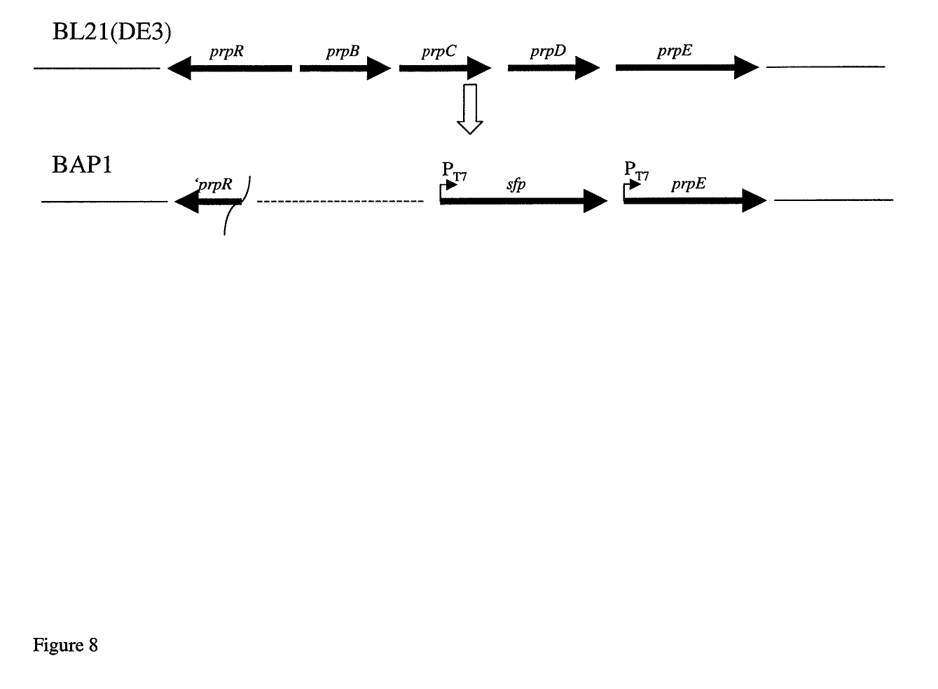

FIG. 9 shows the production of 6dEB in E. coli. Cellular protein content and 6dEB concentration are plotted versus time.

Modes of Carrying Out the Invention

With regard to one aspect of the invention, in the illustrative example below, E. coli is modified to effect the production of 6-dEB, the polyketide precursor of erythromycin. The three proteins required for this synthesis, DEBS1, DEBS2 and DEBS3 are known and the genes encoding them have been cloned and sequenced. However, a multiplicity of additional PKS genes have been cloned and sequenced as well, including those encoding enzymes which produce the polyketide precursors of avermectin, oleandomycin, epothilone, megalomycin, picromycin, FK506, FK520, rapamycin, tylosin, spinosad, and many others. In addition, methods to modify native PKS genes so as to alter the nature of the polyketide produced have been described. Production of hybrid modular PKS proteins and synthesis systems is described and claimed in U.S. Pat. No. 5,962,290. Methods to modify PKS enzymes so as to permit efficient incorporation of diketides is described in U.S. Pat. No. 6,080,555. Methods to modify PKS enzymes by mixing and matching individual domains or groups of domains is described in U.S. Ser. No. 09/073,538. Methods to alter the specificity of modules of modular PKS's to incorporate particular starter or extender units are described in U.S. Ser. No. 09/346,860, now allowed. Improved methods to prepare diketides for incorporation into polyketides is described in U.S. Ser. No. 09/492,733. Methods to mediate the synthesis of the polyketide chain between modules are described in U.S. Ser. No. 09/500,747. The contents of the foregoing patents and patent applications are incorporated herein by reference.

Thus, a selected host may be modified to include any one of many possible polyketide synthases by incorporating therein appropriate expression systems for the proteins included in such synthases. Either complete synthases or partial synthases may be supplied depending on the product desired. If the host produces polyketide synthase natively, and a different polyketide from that ordinarily produced is desired, it may be desirable to delete the genes encoding the native PKS. Methods for such deletion are described in U.S. Pat. No. 5,830,750, which is incorporated herein by reference.

For hosts which do not natively produce polyketides, the enzymes that tailor polyketide synthases may be lacking or deficient, so that in addition to supplying the expression systems for the polyketide synthases themselves, it may be necessary to supply an expression system for these enzymes. One enzyme which is essential for the activity of PKS is a phosphopantetheinyl transferase. The genes encoding these transferases have been cloned and are available. These are described in U.S. patent application Ser. No. 08/728,742, which is now published, for example, in Canadian application 2,232,230. The contents of these documents are incorporated herein by reference.

Depending on the host selected, such hosts may natively include genes which produce proteins that catabolize desired starter and/or extender units. One example includes the prp operon wherein the proteins encoded by subunits A-D catabolize exogenous propionate. The enzyme encoded by prp E is desirable however as it is a propionyl CoA synthetase. The portions of the operon encoding catabolizing enzymes are advantageously disabled in modifying E. coli. Similar operons in other hosts may be disabled as needed.

An assay can be used to determine polyketide production in a cell that is unable to carry out propionate catabolism or anabolism by adding labeled propionate and separating it from polyketide that has been produced.

In general, in all cases, enzymes that enhance the production of starter and/or extender units, and any enzymes required for activation of these production enzymes need to be incorporated in the cells by modifying them to contain expression systems for these proteins.

In one embodiment of this aspect, advantage is taken of the matABC operon, which was recently cloned from Rhizobium trifoli (An, J. H., et al., Eur. J Biochem. (1988) 15:395-402). There are three proteins encoded by this operon.

MatA encodes a malonyl-CoA decarboxylase, which normally catalyzes the reaction: malonyl-CoA→acetyl-CoA+CO₂.

MatB encodes a malonyl-CoA synthetase which catalyzes the reaction: malonic acid+CoASH→malonyl-CoA (in an ATP dependent reaction).

MatC encodes a malonate transporter which is believed to be responsible for transport of malonic acid across the cell membrane.

These enzymes are demonstrated herein to be somewhat promiscuous with respect to substrate in their ability to catalyze the reactions shown. Thus, in addition to malonyl-CoA and malonic acids (for MatA and MatB respectively) as substrates, these enzymes can also utilize methylmalonyl-CoA and methylmalonic acid; ethylmalonyl-CoA and ethylmalonic acid; propylmalonyl-CoA and propylmalonic acid and the like. Thus, these enzymes can be used to provide a variety of starter and extender units for synthesis of desired polyketides. Homologs of this operon are also contemplated.

In another embodiment of this aspect, homologs of matB and matC derived from S. coelicolor (GenBank accession No. AL 163003) can be used.

Also useful in supplying substrates for extender units is the gene encoding propionyl CoA carboxylase. This carboxylase enzyme is a dimer encoded by the pccB and accA2 genes which have been characterized from Streptomyces coelicolor A3 by Rodriguez, E., et al., Microbiology (1999) 145:3109-3119. Methods of making 2S-methylmalonyl CoA using homologs of pccA or pccB genes is also contemplated. A biotin ligase is needed for activation of these proteins. The typical substrate for this enzyme is propionyl-CoA which is then converted to methylmalonyl-CoA; a reaction which is summarized as propionyl-CoA+CO₂→-methylmalonyl-CoA (an ATP dependent reaction).

Other acyl-CoA substrates may also be converted to the corresponding malonyl-CoA products.

In addition to providing modified host cells that are efficient in producing polyketides, the polyketide synthases, their activation enzymes, and enzymes which provide starter and/or extender units can be used in in vitro systems to produce the desired polyketides. For example, the enzymes malonyl-CoA decarboxylase and/or malonyl-CoA synthetase such as those encoded by the matABC operon and/or propionyl-CoA carboxylase such as that encoded by the pccB and accA2 genes can be used in in vitro cultures to convert precursors to suitable extender and starter units for a desired PKS to effect synthesis of a polyketide in a cell-free or in in vitro cell culture system. Purified MatB is particularly advantageously used for the preparative cell free production of polyketides, since CoA thioesters are the most expensive components in such cell-free synthesis systems. Alternatively, as set forth above, these genes are used (in any suitable combination) in a general strategy for production by cells in culture of these substrates. MatB and MatC can be used to effect production of any alpha-carboxylated CoA thioester where the corresponding free acid can be recognized as a substrate by MatB. The MatA protein may also be used to supplement in vitro or in vivo levels of starter units such as acetyl-CoA and propionyl-CoA. The genes encoding propionyl-CoA carboxylase can also be used to provide the enzyme to synthesize suitable extender units in vivo. Either an E. coli cell (or other organism) that makes 2S-methylmalonyl CoA or an E. coli cell (or other organism) that overexpresses a biotin ligase (birA) is also contemplated.

Organisms, preferably E. coli, that contain expression systems to make other polyketide intermediates such as ethylmalonyl CoA or methoxymalonyl CoA are also contemplated.

Thus, the invention includes a method to enhance the production of a polyketide, including a complete polyketide in a microbial host, which method comprises providing said host with an expression system for an enzyme which enhances the production of starter and/or extender units used in constructing the polyketide. A “complete” polyketide is a polyketide which forms the basis for an antibiotic, such as the polyketides which are precursors to erythromycin, megalomycin, and the like. The enzymes include those encoded by the matABC operon and their homologs in other organisms as well as the pccB and accA2 genes encoding propionyl carboxylase and their homologs in other organisms. In another aspect, the invention is directed to a method of enhancing production of polyketides in cell-free systems by providing one or more of these enzymes to the cell-free system.

The invention is also directed to cells modified to produce the enzymes and to methods of producing polyketides using these cells, as well as to methods of producing polyketides using cell-free systems.

The invention also includes a method to enhance polyketide production in a microbial system by supplementing the medium with a substrate for an endogenous enzyme which converts this substrate to a starter or extender unit.

The invention also includes a method to produce polyketides in microbial hosts containing modifications to assist polyketide production, such as disarming of the endogenous genes which encode proteins for catabolism of required substrates, by supplying these cells with synthetic precursors, such as diketide precursors.

The polyketide produced may be one normally produced by the PKS and may exist in nature; in this case the presence of the gene encoding the starter/extender production-enhancing enzyme in vivo or of the enzyme itself in cell free systems may simply enhance the level of production. In addition, the PKS may be a modified PKS designed to produce a novel polyketide, whose production may be enhanced in similar fashion. Because of the ability of the enzymes described herein to accept a wide range of substrates, extender units and starter units can be provided based on a wide range of readily available reagents. As stated above, diketide starting materials may also be supplied.

The invention thus also includes the various other modifications of microbial hosts described above to permit or enhance their production of polyketides and to methods of producing polyketides using such hosts.

The ability to modify hosts such as E. coli and other procaryotes such as Bacillus to permit production of polyketides in such hosts has numerous advantages, many of which reside in the inherent nature of E. coli. One important advantage resides in the ease with which E. coli can be transformed as compared to other microorganisms which natively produce polyketides. One important application of this transformation ease is in assessing the results of gene shuffling of polyketide synthases. Thus, an additional aspect of the invention is directed to a method to assess the results of polyketide synthase gene shuffling which method comprises transfecting a culture of the E. coli modified according to the invention with a mixture of shuffled polyketide synthases and culturing individual colonies. Those colonies which produce polyketides contain successfully shuffled genes.

In addition to modifying microbial hosts, especially procaryotic hosts, to produce polyketides, these hosts may further be modified to produce the enzymes which “tailor” the polyketides and effect their conversion to antibiotics. Such tailoring reactions include glycosylation, oxidation, hydroxylation and the like. Organisms, preferably E. coli, which are modified to contain one or more polyketide modification enzymes, such as those relating to p450, sugar biosynthesis and transfer, and methyl transferase are also contemplated.

To effect production of the polyketides in a microbial host, it is preferable to permit substantial growth of the culture prior to inducing the enzymes which effect the synthesis of the polyketides. Thus, in hosts which do not natively produce polyketides, the required expression systems for the PKS genes are placed under control of an inducible promoter, such as the T7 promoter which is induced by isopropyl-β-D-thiogalactopyranoside (IPTG). There is a plethora of suitable promoters which are inducible in a variety of such microbial hosts. Other advantageous features of the modified host, such as the ability to synthesize starters or extenders, may also be under inducible control. Finally, precursors to the starting materials for polyketide synthase may be withheld until synthesis is desired. Thus, for example, if the starting materials are derived from propionate, propionate can be supplied at any desired point during the culturing of the cells. If a diketide or triketide starting material is used, this too can be withheld until the appropriate time. Prior to addition of the precursor, a minimal medium may be used and alternate carbon sources employed to supply energy and materials for growth.

As described above, the invention provides methods for both in vitro and in vivo synthesis of any arbitrarily chosen polyketide where the in vivo synthesis may be conducted in any microbial, especially procaryotic host. The procaryotic host is typically of the genus Bacillus, Pseudomonas, Flavobacterium, or more typically Escherichia, in particular E. coli. Whether in vitro or in vivo synthesis is employed, it may be necessary to supply one or more of a suitable polyketide synthase (which may be native or modified), one or more enzymes to produce starter and/or extender units, typically including converting the free acid to the CoA derivative, and, if the foregoing enzymes are produced in a host, tailoring enzymes to activate them. In addition, for in vivo synthesis, it may be necessary to disarm catabolic enzymes which would otherwise destroy the appropriate starting materials.

With respect to production of starting materials, the genes of the matABC operon and the genes encoding propionyl carboxylase can be employed to produce their encoded proteins for use in cell free polyketide synthesis and also to modify recombinant hosts for production of polyketides in cell culture. These genes and their corresponding encoded products are useful to provide optimum levels of substrates for polyketide synthase in any host in which such synthesis is to be effected. The host may be one which natively produces a polyketide and its corresponding antibiotic or may be a recombinantly modified host which either does not natively produce any polyketide or which has been modified to produce a polyketide which it normally does not make. Thus, microorganism hosts which are useable for the synthesis of polyketides include various strains of Streptomyces, in particular S. coelicolor and S. lividans, various strains of Myxococcus, industrially favorable hosts such as E. coli, Bacillus, Pseudomonas or Flavobacterium, and other microorganisms such as yeast. These genes and their corresponding proteins are useful in adjusting substrate levels for polyketide synthesis generally.

Substrate Specificity and Polyketide Design

These genes and their products are particularly useful because of the ability of the enzymes to utilize a range of starting materials. Thus, in general, propionyl carboxylase converts a thioester of the formula R₂—CH—CO—SCoA, where each R is H or an optionally substituted alkyl or other optionally substituted hydrocarbyl group to the corresponding malonic acid thioester of the formula R₂C(COOH)COSCoA. Other thioesters besides the natural co-enzyme A thioester may also be used such as the N-acyl cysteamine thioesters. Similarly, the product of the matB gene can convert malonic acid derivatives of the formula R₂C(COOH)₂ to the corresponding acyl thioester, where each R is independently H or optionally substituted hydrocarbyl. A preferred starting material is that wherein R is alkyl (1-4C), preferably RCH(COOH)₂. For in vivo systems, it may be advantageous to include the matC gene to ensure membrane transport of the starting malonic acid related material. The matA gene encodes a protein which converts malonyl-CoA substrates of the formula R₂C(COOH)COSCoA to the corresponding acyl-CoA of the formula R₂CHCOSCoA, where R is defined as above, for use as a starter unit.

Typically, the hydrocarbyl groups referred to above are alkyl groups of 1-8C, preferably 1-6C, and more preferably 1-4C. The alkyl groups may be straight chain or branch chain, but are preferably straight chain. The hydrocarbyl groups may also include unsaturation and may further contain substituents such as halo, hydroxyl, methoxyl or amino or methyl or dimethyl amino. Thus, the hydrocarbyl groups may be of the formula CH₃CHCHCH₂; CH₂CHCH₂; CH₃OCH₂CH₂CH₂; CH₃CCCH₂; CH₃CH₂CH₂CH₂CH₂; and the like.

The substituted alkyl groups are also 1-8C in the backbone chain, preferably 1-6C and more preferably 1-4C. The alkenyl and alkynyl hydrocarbyl groups contain 2-8C, preferably 2-6C, and more preferably 2-4C and may also be branched or straight chain, preferably straight chain.

Further variability can be obtained by supplying as a starting material a suitable diketide. The diketide generally of the formulas such as those set forth in U.S. Ser. No. 09/311,756 filed May 14, 1999 and incorporated herein by reference. A variety of substituents can then be introduced. Thus, the diketide will be of the general formula R′CH₂CHOHCR₂COSNAc wherein R is defined as above, and R′ can be alkyl, 1-8C, aryl, aryl alkyl, and the like. SNAc represents a thioester of N-acetyl cysteamine, but alternative thioesters could also be used.

For either in vivo or in vitro production of the polyketides, acyl transferase domains with desired specificities can be incorporated into the relevant PKS. Methods for assuring appropriate specificity of the AT domains is described in detail in U.S. patent application Ser. No. 09/346,860 filed Jul. 2, 1999, the contents of which are incorporated herein by reference, to describe how such domains of desired specificity can be created and employed. Also relevant to the use of these enzymes in vitro or the genes in vivo are methods to mediate polyketide synthase module effectiveness by assuring appropriate transfer of the growing polyketide chain from one module to the next. Such methods are described in detail in U.S. Ser. No. 09/500,747 filed Feb. 9, 2000, the contents of which are incorporated herein by reference for this description.

As a preliminary matter in determining which substituted benzoates can serve as starter units, adenylation and thiolation activities of the loading module were reconstituted in vitro and shown to be independent of coenzyme A, countering literature proposals that the loading module is a coenzyme A ligase as shown in Example 7. Kinetic parameters for covalent arylation of the loading module were measured directly for the unnatural substrates benzoate (B) and 3-hydroxybenzoate (3-HB) as described in Example 8. This analysis was extended through competition experiments to determine the relative rates of incorporation of a series of substituted benzoates as described in Examples 9 and 10. The results in the examples show that the loading module can accept a variety of substituted benzoates, although it exhibits a preference for the 3-, 5-, and 3,5-disubstituted benzoates that most closely resemble its biological substrate. The remarkable substrate tolerance of the loading module of rifamycin synthetase suggests that the module is useful as a tool for generating substituted derivatives of natural products.

Substituted benzoates are defined as benzoate molecules that include any substituent or substituents. Benzoate substrates is a subset of substituted benzoates that primes an A-T didomain of a rifamycin synthase or otherwise can be incorporated as a starter unit into a loading module or as an extender unit into a module of a synthase or a synthetase. Preferably the benzoate substrates include 3-, 5-, and 3,5-disubstituted benzoates. More preferably, the benzoates are selected from the group consisting of 2-aminobenzoate, 3-aminobenzoate, 4-aminobenzoate, 3-amino-5-hydroxybenzoate, 3-amino-4-hydroxybenzoate, 4-amino-2-hydroxybenzoate, 3-bromobenzoate, 3-chlorobenzoate, 3,5-diaminobenzoate, 3,5-dibromobenzoate, 3,5-dichlorobenzoate, 3,5-difluorobenzoate, 2,3-dihydroxybenzoate, 3,5-dihydroxybenzoate, 3,5-dinitrobenzoate, 3-fluorobenzoate, 2-hydroxybenzoate, 3-hydroxybenzoate, 4-hydroxybenzoate, 3-methoxybenzoate, 3-nitrobenzoate, and 3-sulfobenzoate.

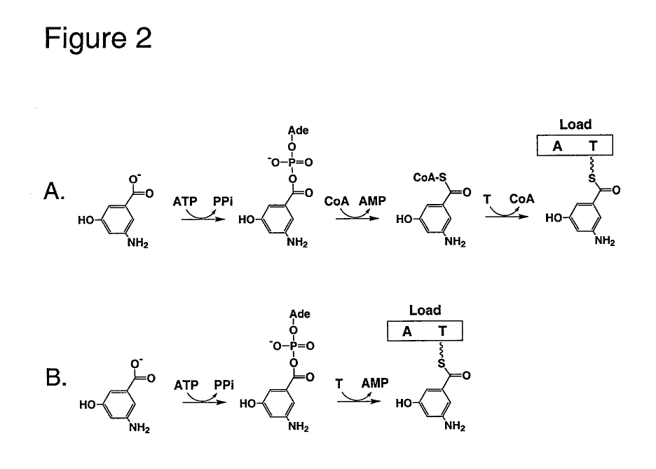

The observation that CoA is not required for arylation of the T domain and that benzoyl-CoA is not a competent intermediate in this process establishes the loading module of rifamycin synthetase as an NRPS-like A-T didomain (FIG. 2B).

The conclusion that the loading module of rifamycin synthetase functions as an NRPS-like A-T didomain has implications for other systems. Biosynthetic gene clusters for rapamycin (Lowden, P. A. S., et al. (1996) Anges. Chem. Int. Ed. Engl. 35, 2249-2251), FK506 (Motamedi, H., et al. (1998) Eur. J. Biochem. 256, 528-534), ansatrienin (Chen, S., et al. (1999) Eur. J. Biochem. 261, 98-107, FK520 (Wu, K., et al. (2000) Gene 251, 81-90), microcystin (Tillett, D., et al. (2000) Chem. Biol. 7, 753-764), and pimaricin (Aparicio, J. F., et al. (2000) Chem. Biol. 7, 895-905) all encode loading modules with homology to the A-T didomain of rifamycin synthetase. However, several of these systems have been proposed to be primed by an activated CoA substrate, presumably generated via a CoA ligase mechanism analogous to that shown in FIG. 2A. (Schwecke, T., et al. (1995) Proc. Natl. Acad. Sci. USA 92, 7839-7843; Motamedi, H., et al. (1998) Eur. J. Biochem. 256, 528-534; Moore, R. E., et al. (1991) J. Am. Chem. Soc. 113, 5083-5084.) A more likely mechanism for priming of these systems is the adenylation-thiolation mechanism operative for rifamycin synthetase.

Although the mechanisms shown in FIG. 2 are distinct, the chemistries involved are essentially the same. In both cases activation of AHB occurs via the aryl-adenylate, and the only difference is whether or not there is intermediate transfer of AHB to CoA prior to arylation of the T domain. Because the phosphopantetheine cofactor of the T domain is derived from CoA, the thiol nucleophiles of the T domain and CoA are chemically equivalent. Therefore, it is not difficult to envision how an enzyme could evolve from a CoA ligase into an A-T didomain, simply by covalent incorporation of the nucleophilic end of CoA as a phosphopantetheine cofactor. There is presumably an advantage to covalently tethering the aryl substrate moiety to the synthetase via the T domain instead of noncovalently binding it as the aryl-CoA. Nevertheless, aryl-CoA ligases are known to be involved in polyketide synthesis in the plant kingdom (see, for example, Beerhues, L. (1996) FEBS Lett. 383, 264-266; Barillas, W., et al. (2000) Biol. Chem. 381, 155-160), and benzoyl-CoA appears to be a substrate of the iterative type II PKS that produces enterocin (Hertweck, C., et al. (2000) Tetrahedron 56, 9115-9120).

Prior to this investigation, AHB, 3-HB, and 3,5-dihydroxybenzoate were known to be substrates of the A-T didomain (Hunziker, D., et al. (1998) J. Am. Chem. Soc. 120, 1092-1093). Eleven additional substrates, including benzoate (B), have been identified herein (Table 1). Previous work suggests that the substrate tolerance of the A-T didomain of rifamycin synthetase for alternative substituted benzoates is shared to a degree by related bacterial benzoyl-CoA ligases (Geissler, J. F., et al. (1988) J. Bact. 170, 1709-1714; Altenschmidt, U., (1991) J. Bact. 173, 5494-5501); and EntE (Rusnak, R., et al. (1989) Biochemistry 28, 6827-6835), a stand-alone A domain that is a component of the enterobactin synthetase. These proteins are able to accept several alternative substituted benzoates, in addition to their biological substrates.

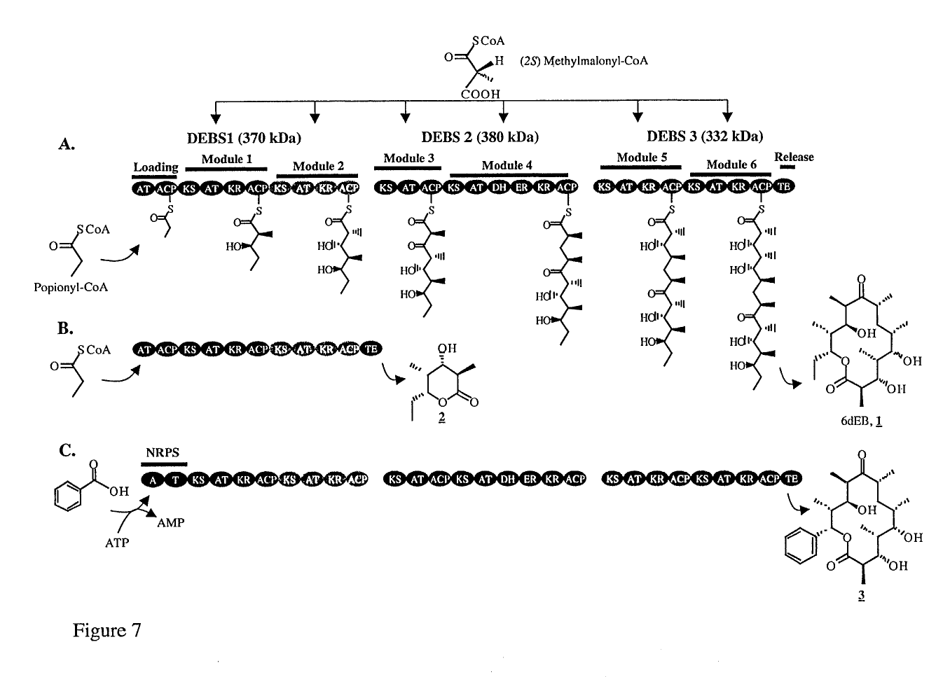

Although analysis of the substrate specificity results for the A-T didomain at a detailed molecular level awaits a crystal structure of this loading module, some preliminary observations can be made based on the substrate screening results and the relative reactivity data in Table 1. With the exception of 2-aminobenzoate and B, only benzoates with 3-, 5-, or both 3- and 5-substituents are substrates for the A-T didomain. Binding sites that accommodate the 3-amino- and 5-hydroxy- substituents of the biological substrate AHB can apparently also accommodate alternative substituents at these positions. 3-Sulfobenzoate, 3-nitrobenzoate, and 3,5-dinitrobenzoate were likely rejected as substrates for steric reasons (FIG. 7), since both sulfo- and nitro-substituents are significantly larger than the amino- and hydroxy-substituents of AHB. In this regard, it is surprising that 3-methoxybenzoate is accepted as a substrate, albeit a poor one, since the methoxy- substituent is also significantly larger than either substituent of AHB. The 3-fluoro- and 3,5-difluorobenzoates are discriminated against by factors of 5 and 30 with respect to their chlorinated and brominated counterparts (Table 1). Changes in the electronic properties of the aromatic ring upon fluorination may account for these differences. Phenylacetate and 3-hydroxyphenylacetate do not appear to be utilized as substrates by the A-T didomain, despite the reactivity of the corresponding benzoates, B and 3-HB (Table 1). This result suggests that the register of the carboxylate is a determinant of its reactivity, as the carboxylate of the phenylacetates is displaced by one methylene group relative to the benzoates. It should be noted that substituted benzoates were targeted as putative substrates in this study; the possibility that the tolerance of the A-T didomain for substituted benzoates extends to other types of aromatic substrates (e.g., heterocycles) remains to be tested.

The remarkable substrate tolerance of the loading module of rifamycin synthetase for substituted benzoates has implications for the production of unnatural natural products through protein engineering. The endogenous loading module of 6-deoxyerythronolide B PKS was recently replaced by the loading module of the avermectin PKS, and the resulting hybrid synthase produced erythromycin derivatives that had incorporated branched starter units characteristic of the avermectin family (Marsden, A. F., et al. (1998) Science 279, 199-202). Similarly, exploiting the priming promiscuity of the A-T didomain of rifamycin synthetase by appending it to other synthases or synthetases, with the goal of generating substituted derivatives of the original products is contemplated according to the invention.

Finally, this initial characterization of the loading module of rifamycin synthetase as an NRPS-like A-T didomain sets the stage for investigation of the hybrid NRPS/PKS interface in this system. Biochemical studies that combine the NRPS-like loading module and PKS module 1 of rifamycin synthetase (in cis or in trans) should allow functional and structural questions regarding NRPS/PKS biosynthetic interfaces to be addressed.

The nucleotide sequences encoding a multiplicity of PKS permits their use in recombinant procedures for producing a desired PKS and for production of the proteins useful in postmacrolide conversions, as well as modified forms thereof. For example, the nucleotide sequences for genes related to the production of erythromycin is disclosed in U.S. Pat. Nos. 6,004,787 and 5,998,194; for avermectin in U.S. Pat. No. 5,252,474; for FK506 in U.S. Pat. No. 5,622,866; for rifamycin in WO98/7868; for spiramycin in U.S. Pat. No. 5,098,837. These are merely examples. Portions of, or all of, the desired coding sequences can be synthesized using standard solid phase synthesis methods such as those described by Jaye et al., J Biol Chem (1984) 259:6331 and which are available commercially from, for example, Applied Biosystems, Inc.

A portion of the PKS which encodes a particular activity can be isolated and manipulated, for example, by using it to replace the corresponding region in a different modular PKS. In addition, individual modules of the PKS may be ligated into suitable expression systems and used to produce the portion of the protein encoded by the open reading frame and the protein may then be isolated and purified, or which may be employed in situ to effect polyketide synthesis. Depending on the host for the recombinant production of the module or an entire open reading frame, or combination of open reading frames, suitable control sequences such as promoters, termination sequences, enhancers, and the like are ligated to the nucleotide sequence encoding the desired protein. Suitable control sequences for a variety of hosts are well known in the art.

The availability of these nucleotide sequences expands the possibility for the production of novel polyketides and their corresponding antibiotics using host cells modified to contain suitable expression systems for the appropriate enzymes. By manipulating the various activity-encoding regions of a donor PKS by replacing them into a scaffold of a different PKS or by forming hybrids instead of or in addition to such replacements or other mutagenizing alterations, a wide variety of polyketides and corresponding antibiotics may be obtained. These techniques are described, for example, in U.S. Ser. No. 09/073,538 filed May 6, 1998 and incorporated herein by reference.

A polyketide synthase may be obtained that produces a novel polyketide by, for example, using the scaffolding encoded by all or the portion employed of a natural synthase gene. The synthase will contain at least one module that is functional, preferably two or three modules, and more preferably four or more modules and contains mutations, deletions, or replacements of one or more of the activities of these functional modules so that the nature of the resulting polyketide is altered. This description applies both at the protein and genetic levels. Particularly preferred embodiments include those wherein a KS, AT, KR, DH or ER has been deleted or replaced by a version of the activity from a different PKS or from another location within the same PKS. Also preferred are derivatives where at least one noncondensation cycle enzymatic activity (KR, DH or ER) has been deleted or wherein any of these activities has been mutated so as to change the ultimate polyketide synthesized.

Thus, in order to obtain nucleotide sequences encoding a variety of derivatives of the naturally occurring PKS, and a variety of polyketides, a desired number of constructs can be obtained by “mixing and matching” enzymatic activity-encoding portions, and mutations can be introduced into the native host PKS gene cluster or portions thereof.

Mutations can be made to the native sequences using conventional techniques. The substrates for mutation can be an entire cluster of genes or only one or two of them; the substrate for mutation may also be portions of one or more of these genes. Techniques for mutation include preparing synthetic oligonucleotides including the mutations and inserting the mutated sequence into the gene encoding a PKS subunit using restriction endonuclease digestion (See, e.g., Kunkel, T. A. Proc Natl Acad Sci USA (1985) 82:448; Geisselsoder et al. BioTechniques (1987) 5:786.) or by a variety of other art-known methods.

Random mutagenesis of selected portions of the nucleotide sequences encoding enzymatic activities can also be accomplished by several different techniques known in the art, e.g., by inserting an oligonucleotide linker randomly into a plasmid, by irradiation with X-rays or ultraviolet light, by incorporating incorrect nucleotides during in vitro DNA synthesis, by error-prone PCR mutagenesis, by preparing synthetic mutants or by damaging plasmid DNA in vitro with chemicals.

In addition to providing mutated forms of regions encoding enzymatic activity, regions encoding corresponding activities from different PKS synthases or from different locations in the same PKS synthase can be recovered, for example, using PCR techniques with appropriate primers. By “corresponding” activity encoding regions is meant those regions encoding the same general type of activity—e.g., a ketoreductase activity in one location of a gene cluster would “correspond” to a ketoreductase-encoding activity in another location in the gene cluster or in a different gene cluster; similarly, a complete reductase cycle could be considered corresponding—e.g., KR /DH/ER would correspond to KR alone.

If replacement of a particular target region in a host polyketide synthase is to be made, this replacement can be conducted in vitro using suitable restriction enzymes or can be effected in vivo using recombinant techniques involving homologous sequences framing the replacement gene in a donor plasmid and a receptor region in a recipient plasmid. Such systems, advantageously involving plasmids of differing temperature sensitivities are described, for example, in PCT application WO 96/40968.

Finally, polyketide synthase genes, like DNA sequences in general, in addition to the methods for systematic alteration and random mutagenesis outlined above, can be modified by the technique of “gene shuffling” as described in U.S. Pat. No. 5,834,458, assigned to Maxygen, and U.S. Pat. Nos. 5,830,721, 5,811,238 and 5,605,793, assigned to Affymax. In this technique, DNA sequences encoding bPKS are cut with restriction enzymes, amplified, and then re-ligated. This results in a mixture of rearranged genes which can be assessed for their ability to produce polyketides. The ability to produce polyketides in easily transformed hosts, such as E. coli, makes this a practical approach.

There are five degrees of freedom for constructing a polyketide synthase in terms of the polyketide that will be produced. First, the polyketide chain length will be determined by the number of modules in the PKS. Second, the nature of the carbon skeleton of the PKS will be determined by the specificities of the acyl transferases which determine the nature of the extender units at each position—e.g., malonyl, methyl malonyl, or ethyl malonyl, etc. Third, the loading domain specificity will also have an effect on the resulting carbon skeleton of the polyketide. Thus, the loading domain may use a different starter unit, such as acetyl, propionyl, butyryl and the like. Fourth, the oxidation state at various positions of the polyketide will be determined by the dehydratase and reductase portions of the modules. This will determine the presence and location of ketone, alcohol, double bonds or single bonds in the polyketide. Finally, the stereochemistry of the resulting polyketide is a function of three aspects of the synthase. The first aspect is related to the AT/KS specificity associated with substituted malonyls as extender units, which affects stereochemistry only when the reductive cycle is missing or when it contains only a ketoreductase since the dehydratase would abolish chirality. Second, the specificity of the ketoreductase will determine the chirality of any β-OH. Finally, the enoyl reductase specificity for substituted malonyls as extender units will influence the result when there is a complete KR/DH/ER available.

One useful approach is to modify the KS activity in module 1 which results in the ability to incorporate alternative starter units as well as module 1 extended units. This approach was illustrated in PCT application U.S./96111317, incorporated herein by reference, wherein the KS-I activity was inactivated through mutation. Polyketide synthesis is then initiated by feeding chemically synthesized analogs of module 1 diketide products. The methods of the invention can then be used to provide enhanced amount of extender units.

Modular PKSs have relaxed specificity for their starter units (Kao et al. Science (1994), supra). Modular PKSs also exhibit considerable variety with regard to the choice of extender units in each condensation cycle. The degree of β-ketoreduction following a condensation reaction has also been shown to be altered by genetic manipulation (Donadio et al. Science (1991), supra; Donadio, S. et al. Proc Natl Acad Sci USA (1993) 90:7119-7123). Likewise, the size of the polyketide product can be varied by designing mutants with the appropriate number of modules (Kao, C. M. et al. J Am Chem Soc (1994) 116:11612-11613). Lastly, these enzymes are particularly well-known for generating an impressive range of asymmetric centers in their products in a highly controlled manner. The polyketides and antibiotics produced by the methods of the present invention are typically single stereoisomeric forms. Although the compounds of the invention can occur as mixtures of stereoisomers, it is more practical to generate individual stereoisomers using the PKS systems.

The polyketide products of the PKS may be further modified, typically by hydroxylation, oxidation and/or glycosylation, in order to exhibit antibiotic activity.

Methods for glycosylating the polyketides are generally known in the art; the glycosylation may be effected intracellularly by providing the appropriate glycosylation enzymes or may be effected in vitro using chemical synthetic means as described in U.S. Ser. No. 09/073,538 incorporated herein by reference.

The antibiotic modular polyketides may contain any of a number of different sugars, although D-desosamine, or a close analog thereof, is most common. For example, erythromycin, picromycin, narbomycin and methymycin contain desosamine. Erythromycin also contains L-cladinose (3-O-methyl mycarose). Tylosin contains mycaminose (4-hydroxy desosamine), mycarose and 6-deoxy-D-allose. 2-acetyl-1-bromodesosamine has been used as a donor to glycosylate polyketides by Masamune et al. J Am Chem Soc (1975) 97:3512, 3513. Other, apparently more stable, donors include glycosyl fluorides, thioglycosides, and trichloroacetimidates; Woodward, R. B. et al. J Am Chem Soc (1981) 103:3215; Martin, S. F. et al. Am Chem Soc (1997) 119:3193; Toshima, K. et al. J Am Chem Soc (1995) 117:3717; Matsumoto, T. et al. Tetrahedron Lett (1988) 29:3575. Glycosylation can also be effected using the macrolides as starting materials and using mutants of S. erythraea that are unable to synthesize the macrolides to make the conversion.

In general, the approaches to effecting glycosylation mirror those described above with respect to hydroxylation. The purified enzymes, isolated from native sources or recombinantly produced may be used in vitro. Alternatively, glycosylation may be effected intracellularly using endogenous or recombinantly produced intracellular glycosylases. In addition, synthetic chemical methods may be employed.

If the hosts ordinarily produce polyketides, it may be desirable to modify them so as to prevent the production of endogenous polyketides by these hosts. Such hosts have been described, for example, in U.S. Pat. No. 5,672,491, incorporated herein by reference, which describes S. coelicolor CH999 used in the examples below. In such hosts, it may not be necessary to provide enzymatic activity for posttranslational modification of the enzymes that make up the recombinantly produced polyketide synthase; these hosts generally contain suitable enzymes, designated holo-ACP synthases, for providing a pantetheinyl residue needed for functionality of the synthase. However, in hosts such as yeasts, plants, or mammalian cells which ordinarily do not produce polyketides, it may be necessary to provide, also typically by recombinant means, suitable holo-ACP synthases to convert the recombinantly produced PKS to functionality. Provision of such enzymes is described, for example, in PCT application WO 98/27203, incorporated herein by reference.

Again, depending on the host, and on the nature of the product desired, it may be necessary to provide “tailoring enzymes” or genes encoding them, wherein these tailoring enzymes modify the macrolides produced by oxidation, hydroxylation, glycosylation, and the like.

The encoding nucleotide sequences are operably linked to promoters, enhancers, and/or termination sequences which operate to effect expression of the encoding nucleotide sequence in host cells compatible with these sequences; host cells modified to contain these sequences either as extrachromosomal elements or vectors or integrated into the chromosome, and methods to produce PKS and post-PKS enzymes as well as polyketides and antibiotics using these modified host cells. Multiple vector systems for use in organisms such as E. coli are contemplated.

The vectors used to perform the various operations to replace the enzymatic activity in the host PKS genes or to support mutations in these regions of the host PKS genes may be chosen to contain control sequences operably linked to the resulting coding sequences in a manner that expression of the coding sequences may be effected in a appropriate host. However, simple cloning vectors may be used as well.

Particularly useful control sequences are those which themselves, or using suitable regulatory systems, activate expression during transition from growth to stationary phase in the vegetative mycelium. The system contained in the illustrative plasmid pRM5, i.e., the actI/actIII promoter pair and the actII-ORF4, an activator gene, is particularly preferred. Particularly preferred hosts are those which lack their own means for producing polyketides so that a cleaner result is obtained. Illustrative host cells of this type include the modified S. coelicolor CH999 culture described in PCT application WO 96/40968 and similar strains of S. lividans.

Methods for introducing the recombinant vectors of the present invention into suitable hosts are known to those of skill in the art and typically include the use of CaCl₂ or other agents, such as divalent cations, lipofection, DMSO, protoplast transformation and electroporation.

As disclosed in Ser. No. 08/989,332 filed Dec. 11, 1997, incorporated herein by reference, a wide variety of hosts can be used, even though some hosts natively do not contain the appropriate post-translational mechanisms to activate the acyl carrier proteins of the synthases. These hosts can be modified with the appropriate recombinant enzymes to effect these modifications.

To demonstrate the power of engineering modular polyketide synthases in a new heterologous system, we attempted to construct a derivative of DEBS in which a PKS module was fused to a nonribosomal peptide synthetase (NRPS)-like module (Mootz, H. D., et al., Curr. Opin. Chem. Biol. 1:543 (1997)). The first module of the rifamycin synthetase has recently been shown to be an NRPS-like module comprised of two domains: an adenylation (A) domain and a thiolation (T) domain (Admiraal, S. J., et al, Biochemistry Submitted). The A domain activates 3-amino-5-hydroxybenzoate (as well as benzoate and several benzoate derivatives, (Admiraal, supra)) in an ATP-dependent reaction, and transfers the aryl adenylate onto the phosphopantetheine arm of the T domain (FIG. 7). This NRPS-like module was fused upstream of the first condensation module of DEBS in lieu of the loading didomain of DEBS (The construction of plasmid pBP165, carrying the rifamycin loading didomain fused to DEBS1 as well as the pccAB genes, is described in Example 11.) In the presence of exogenous propionate and benzoate, the resulting strain of E. coli produced the expected 6dEB analog (compound 3), as confirmed by NMR and mass spectrometry (FIG. 7) (¹³C-NMR (CDCl₃, 500 MHz) δ213.76, 177.43, 79.70, 76.60, 71.24, 37.72 (enriched carbon atoms only). Mass Spectrometry (AP-CI) for expected ¹²C₁₉¹³C₆H₃₈O₆Na: 463.2757; observed: 463.2847.).

In summary, we have demonstrated the feasibility of engineering E. coli to produce complex polyketide natural products. Multiple changes were made to the E. coli genome for relevant 6dEB production, including introduction of the three DEBS genes from Saccharopolyspora erythraea, introduction of the sfp phosphopantetheinyl transferase gene from Bacillus subtilis, introduction of genes encoding a heterodimeric propionyl-CoA carboxylase from Streptomyces coelicolor, deletion of the endogenous prpRBCD genes, and overexpression of the endogenous prpE and birA genes. When gene expression was coordinately induced at low temperature, propionate could be converted into 6dEB by this metabolically engineered cellular catalyst with excellent kinetic parameters. Given the availability of well-established scalable protocols for fermenting E. coli to overproduce bioproducts, the ability to synthesize complex polyketides in this heterologous host bodes well for the practical production of these bioactive natural products. Equally important, as indicated by the hybrid PKS-NRPS described here, it opens the door for harnessing the enormous power of molecular biology in E. coli to engineer modular polyketide synthases using directed and random approaches. As such, organisms such as E. coli that make a hybrid modular polyketide synthases such as one that comprises NRPS and incorporate a variety of benzoate substrates are also contemplated.

Starting Material Enhancement and Variation

Thus, proteins (and their encoding sequences) wherein the proteins catalyze the production of starter and/or extender units can be used to enhance the production of polyketides by providing a considerable variety of these starter and extender units at higher levels than would ordinarily be produced. Because the proteins catalyze reactions using a variety of substrates, they are versatile tools in enhancing the availability of starter and extender units for a wide variety of PKS, whether modified or unmodified. As stated above, particularly useful are the products of the matABC operon (or analogous operons in other species) and the propionic carboxylase encoded by the pccB and accA2 genes (or their analogs in other species). These enzymes and their encoding sequences are useful in view of Applicants' discovery that the matABC operon and the propionic carboxylase-encoding genes provide enzymes which not only carry out the required reactions on a variety of substances, but also do so with the production of products with the stereochemistry required for use in polyketide synthesis.

The ability of the genes described herein to provide appropriate starter and extender units was established as described below.

### EXAMPLE 1

Production of Malonyl CoA and 2S-Methylmalonyl CoA Using the CoA Synthetase

E. coli strain L8 has a temperature-sensitive mutation in the acetyl-CoA carboxylase gene such that malonyl-CoA cannot be produced from acetyl-CoA at 37° C. However, the gene product is able to effect this conversion at 30° C. See Harder, M. E., et al., Proc. Natl. Acad. Sci. (1972) 69:3105-3109. Since acetyl-CoA carboxylase conversion of acetyl-CoA into malonyl-CoA is the only known route for malonyl-CoA production in E. coli, and since malonyl-CoA is essential for fatty acid biosynthesis, this mutant strain of E. coli can grow at 30° C., but not at 37° C. A transformant of L8 carrying the matABC operon is produced by transforming with the plasmid pMATOP2 which contains the matA, matB and matC genes under control of their native promoter and is described in An, J. H., et al., Eur. J Biochem. (1998) 257:395-402. This transformant is still unable to grow at 37° C. in the absence of malonic acid; however, addition of 1-5 mM malonic acid to the medium permits it to grow at this temperature. (In the absence of the plasmid, malonic acid is unable to support growth at 37° C.) The concentration of the extracellular malonic acid is important, however, as increasing the concentration to 40 mM results in an absence of growth, possibly due to a metabolic imbalance caused by overproduction of malonyl CoA in comparison to the amount of coenzyme A available. Lethality was also induced in XL1-Blue (a wild-type strain of E. coli) in the presence of the plasmid carrying the matABC operon and high concentrations of methylmalonic acid.

Nevertheless, the results set forth above demonstrate that the protein encoded by matB produces malonyl-CoA in vivo under physiological conditions as long as free malonic acid is available; and transported into the cells by the protein encoded by matC. Thus, the matBC genes can be used to supplement malonyl-CoA availability in an E. coli cell in which complex polyketides are to be produced by feeding malonic acid.

In addition to converting malonic acid into malonyl-CoA, MatB has also been shown to convert methyhnalonic acid into methylmalonyl-CoA. However the stereochemistry of the resulting product has not been reported. This is important, because modular polyketide synthases are known to only accept one isomer of methylmalonyl-CoA, namely 2S-methyhnalonyl-CoA (Marsden, A. F., et al., Science (1994) 263:378-380). To investigate whether MatB can make the correct isomer of methyhnalonyl-CoA, construct encoding a glutathione-S-transferase fusion (GST-MatB) was used to produce this protein. See An, J. H., et al., Biochem. J (1999) 344:159-166. The GST-MatB protein was purified according to standard protocols as described and mixed with (module 6+TE) of the erythromycin polyketide synthase, also expressed in E. coli as described by Gokhale, R. S., et al., Science (1999) 284:482-485.

In earlier studies, Applicants have established the activity of (module 6+TE) by demonstrating its ability to catalyze the following reaction in vitro.

N-acetylcysteamine thioester of (2S, 3R)-2-methyl-3-hydroxy-pentanoic acid+2 (RS)-methylmalonyl-CoA+NADPH→(2R,3S,4S,5R)-2,4-dimethyl-3,5 -dihydroxy-n-heptanoic acid δ-lactone+NADP⁺.

The methylmalonic thioester product obtained using methylmalonic acid as the substrate for GST-MatB provides the correct stereochemistry to serve as the source of the extender unit in this reaction. More specifically, to generate the substrate for the above polyketide synthesis in situ, the following reaction mixture (containing 6+TE and GST-MatB) was prepared in a reaction buffer of 100 mM Na Phosphate (pH7) buffer, 1 mM ethylenediaminetetraacetic acid (EDTA), 2.5 mM dithiothreitol (DTT) and 20% glycerol:

40 mM methylmalonic acid (pH 6)

16.6 mM MgCl₂

5 mM ATP

5 mM CoASH

13.3 mM NADPH

1 mM N-acetylcysteamine thioester of (2S, 3R)-2-methyl-3-hydroxypentanoic acid (prepared in radioactive form).

After 4 hrs, the reaction was quenched and extracted with ethyl acetate (extracted twice with three times the reaction volume). The samples were dried in vacuo and subjected to thin layer chromatography analysis.

A positive control was performed under identical conditions to those described earlier—i.e., conditions wherein (RS)-methyhnalonyl-CoA was substituted for the combination of methylmalonic acid, MgCl₂, ATP, CoA SH, and GST-MatB. A negative control included all of the components listed above except for the GST-MatB fusion protein. The results demonstrated that the two-enzyme system described above is able to produce the expected product in quantities comparable to the positive control reaction. This confirms that MatB synthesizes the correct isomer of methylmalonyl-CoA.

Thus, MatB/MatC is useful to synthesize both malonyl-CoA and 2S-methylmalonyl-CoA in vivo for polyketide biosynthesis. This is the first instance of engineering E. coli with the ability to produce 2S-methylmalonyl-CoA in vivo under physiological conditions. Moreover, co-expression of matA in vivo should allow conversion of methylmalonyl-CoA into propionyl-CoA, thereby supplementing available sources of this starter unit.

### EXAMPLE 2

Ability of Propionyl CoA Carboxylase to Generate 2S-Methylmalonyl CoA

To utilize the propionyl carboxylase gene from S. coelicolor described above, an E. coli expression host (BL-21 (DE3)) was prepared using the method developed by Hamilton, C. M., et al., J. Bacteriol. (1989) 171:4617-4622. The new strain (BAP1) contains a phosphopantethiene-transferase gene (the sfp gene) from Bacillus subtilis integrated into the prp operon of E. coli. The T7 promoter drives sfp expression. In the recombination procedure, the prpE gene was also placed under control of the T7 promoter, but the rest of the operon was removed. This genetic alteration would ideally provide three features: 1) the expression of the sfp protein needed for post-translational modification of the DEBS and potentially other polyketide synthases (PKSs); 2) the expression of the prpE protein, a putative propionyl-CoA synthetase theoretically capable of ligating CoASH to propionate; and 3) a cellular environment that is no longer able to metabolize propionyl-CoA as a carbon/energy source.

First, it was verified that the BAP1 strain, by virtue of its production of the product of the sfp gene was able to effect phosphopantetheinylation of a PKS produced in these cells. BAP1 was transfected with a plasmid comprising an expression system for module 6+TE and the activity of the module produced was compared to the activity of the module produced recombinantly in BL-21 (DE3) cells where the sfp gene was plasmid borne. These levels were comparable. In contrast, when expressed alone in BL-21 (DE3), module 6+TE showed no activity. Additionally, BAP1 was confirmed for its inability to grow on propionate as a sole carbon source (a property exhibited by E. coli strains such as BL21 (DE3)). BAP1 is a preferred host for the heterologous expression of polyketide synthases in conjunction with enzymes such as MatBC and propionyl-CoA carboxylase.

The propionyl-CoA carboxylase enzyme was expressed in E. coli under the T7 promoter. The product enzyme was able to supply substrate for module 6+TE in vitro. This was demonstrated using the coupling of the methyhnalonyl-CoA thioester product of the propionyl CoA carboxylase enzyme to the N-acetyl cysteamine thioester of (2S,2R)2-methyl-3-hydroxypentanoic acid. The pccB and accA2 genes described above which encode the components of the propionyl-CoA carboxylase, were expressed and the products individually purified according to standard procedures. Initially, the pccB and accA2 subunits were allowed to complex on ice in 150 mM phosphate (pH7) and 300 μg BSA. After 1 hour, the following substrates were added to a volume of 100 μl and incubated for an additional 30 minutes at 30° C.:

1 mM propionyl-CoA

50 mM sodium bicarbonate

3 mM ATP

5 mM MgCl₂

Module 6+TE was then added with the following final set of reagents to give 200 μl total and allowed to react for an additional hour at 30° C.:

10% glycerol

1.25 mM DTT

0.5 mM EDTA

4 mM NADPH

2 mM N-acetylcysteamine thioester of (2S, 3R)-2-methyl-3-hydroxypentanoic acid (prepared in radioactive form).

The reaction was quenched and extracted as described above, and showed formation of expected product. A positive control included racemic malonyl-CoA. When either ATP or sodium bicarbonate was removed from the reaction, no product was formed. The propionyl-CoA carboxylase thus produces a substrate suitable for polyketide synthase activity. This is particularly useful for polyketide production, especially in conjunction with the new expression host mentioned above, BAP1.

The DEBS protein DEBS 1+TE is produced by pRSG32. DEBS1 shows the weakest expression of the three DEBS proteins and, until recently, the enzyme showed no in vitro activity. However, by growing E. coli containing pRSG32 in M9 minimal medium, and inducing protein expression at 22° C., DEBS1+TE activity is now reproducibly observed.

Plasmids pRSG32 (DEBS1+TE) and p132 (a plasmid containing the α and β components of propionyl-CoA carboxylase) were cotransfected into BAP1. Cultures of 10 ml M9 minimal media were grown to mid-log phase levels and concentrated to 1 ml for induction with IPTG and the addition of 0.267 mM ¹⁴C-propionate. The samples were then incubated at 22° C. for 12-15 hours. The culture supernatant was then extracted with ethyl acetate for analytical TLC. A product ran with the expected positive control and this same product was undetectable when using either wild type BL-21 (DE3) or removing p132. thus, the carboxylase forms the correct stereoisomer.

In addition, 100 ml cultures of M9 minimal media containing BAP1 transformed with pRSG32, p132, and pCY214 (a biotin ligase included to aid biotin's attachment to the α subunit of the propionyl-CoA carboxylase) were grown to mid-log phase for induction with IPTG and the addition of 100 mg/L ¹³C-propionate. The activity of the biotinylated subunit (pccA) could be significantly enhanced upon co-expression of the E. coli birA biotin ligase gene. Upon extraction of the culture supernatant and concentration of the sample, ¹³C-NMR confirmed the location of the expected enriched product peaks. A subsequent negative control using BL-21 (DE3) failed to yield the same spectrum. In addition to demonstrating the ability of E. coli to make complex polyketides in vivo, these results also suggest that the prpE protein programmed to express in BAP1 is active.

Alternatively, M9 minimal media cultures of transformed cells were grown at 37° C. to mid-log phase, followed by induction at 22° C. with 0.5-1 mM IPTG, 2.5 g/L arabinose, and 26 mg/L or 250 mg/L [1-¹⁴C]- or [1-¹³C] -propionate, respectively. Regarding the ¹⁴C-1-propionate feeding, individual transformants were inoculated into M9 minimal media cultures with glucose (Maniatis, T., et al., Molecular Cloning: A Laboratory Manual. 1982) in the presence of 50 μg/ml carbenicillin, 25 μg/ml kanamycin, and 17 μg/ml chloramphenicol at 37° C. and 250 rpm. Cultures were grown to mid-log phase (OD₆₀₀=0.6-0.8), cooled at 22° C. for 5 min, and then centrifuged. The cell pellets were resuspended in 1 ml of the remaining supernatant and induced with 1 mM IPTG and 0.25% arabinose (for pCY216). In addition, regarding the ¹⁴C-1-propionate (at 56 mCi/mmol) was added at final concentration of 0.27 mM. The culture was then stirred for an additional 12-15 hrs at 22° C. At this point the culture was centrifuged and 100 μl of the supernatant was extracted (2×) with ethyl acetate (300 μL each time). The extract was dried in vacuo and subjected to TLC analysis. Negative controls included cultures of BAP1/pRSG32/pCY216 and BL21(DE3)/pRSG32/pTR132/pCY216.

Regarding the ¹³C-1-propionate feeding, a single transformant of BAP1/pRSG32/pTR132/pCY216 was used to start a 3 mL LB culture with 100 μg/ml carbenicillin, 50 μg/ml kanamycin, and 34 μ/ml chloramphenicol at 37° C. and 250 rpm. The starter culture was used to inoculate 100 mL M9 minimal media with glucose at the same antibiotic concentrations as above. These cultures were grown at 250 rpm and 37° C. to mid-log phase (OD₆₀₀=0.5-0.7), cooled for 15 minutes in a 22° C. bath, and induced with 500 μM IPTG and 0.25% arabinose. ¹³C-1-propionate was added at 100 mg/L and the cultures were incubated at 22° C. for 12-15 hrs. The sample was then centrifuged and the supernatant extracted twice with 300 ml ethyl acetate. The sample was dried in vacuo, resuspended in CDCl₃, and analyzed via ¹³C-NMR. A negative control was performed with BL21(DE3)/pRSG32/pTR132/pCY216. After 12-48 hours the culture supernatant was extracted and analyzed for formation of the expected triketide lactone (FIG. 7, compound 2) product of DEBS1+TE. Formation of triketide lactone under both feeding conditions confirmed the ability of BAP 1 to produce polyketides.

Construction of plasmids pRSG32, pBP49, pRSG50: Genes encoding DEBS1+TE (pRSG32), DEBS2 (pBP49) and DEBS3 (pRSG50) were cloned into pET21c (Novagen). The DEBS1+TE gene was cloned as the NdeI-EcoRI fragment from pCK12 (6). The DEBS3 gene was cloned as the NdeI-EcoRI fragment from pJRJ10 (Jacobsen, J. R., et al., Biochemistry 37:4928 (1998)). To express the DEBS2 gene, the BsmI-EcoRI fragment from pRSG34 (Gokhale, R. S., et aL, Science 284:482 (1999)), which has been used previously to express module 3+TE, was replaced with a BsmI-EcoRI fragment encoding module 4. The EcoRI site (in bold) was engineered immediately upstream of the stop codon of the DEBS2 gene by modifying the natural sequence to the following: CGGGGGAGAGGACCTGAATTC.

It should be noted that a first attempt was made to express the genes encoding each of the three DEBS proteins, followed by in vitro assays of protein activity. DEBS3, DEBS2 and a variant of DEBS1, DEBS 1+TE were cloned individually into the pET21c expression vector and introduced via transformation into E. coli BL21(DE3) harboring the sfp phosphopantetheinyl transferase gene on pRSG56 (Kao, C. M., et al., J Am. Chem. Soc. 117:9105-9106 (1995), Cortes, J., et al., Science 268:1487-1489 (1995); Lambalot, R. H., et al., Chemistry & Biology 3:923-936 (1996); Gokhale, R. S., et al., Science 284:482-485 (1999)). The expression levels of the three DEBS genes were found to be comparable to those reported earlier from S. erythraea (Caffrey, P., et al., FEBS Letters 304:225-228 (1992)) or S. coelicolor (Pieper, R., et al., Nature 378:263-266 (1995)). Individual transformants were used to start 25 ml LB seed cultures containing 100 μg/ml carbenicillin and 50 μg/ml kanamycin at 250 rpm and 37° C. These cultures were used to inoculate 1 L of LB medium, and the culture was grown under the same conditions. At mid-log phase (OD₆₀₀=0.4-0.8) cells were induced with 1 mM IPTG and transferred to a 30° C. incubator. Cells were harvested after 4-6 hours and their protein content was analyzed via 7.5% SDS-PAGE. The three DEBS proteins were expressed at ca. 1% total cellular protein. However, although DEBS3 was found to be active in these lysates, DEBS1+TE and DEBS2 lacked any detectable activity (DEBS1+TE (Pieper, R., supra) and DEBS3 were assayed as described earlier (Jacobsen, J. R., et al, Biochemistry 37:4928 (1998)). Although an assay for the entire DEBS2 has not yet been developed, the activity of module 3 on this protein can be assayed as described earlier (Gokhale, R. S., supra). Consistent with these results, recombinant DEBS3 could be purified from these lysates using procedures described earlier, (Pohl, N. L., et al., J. Am. Chem. Soc. 120:11206-11207 (1998)), but neither DEBS1+TE nor DEBS2 could be purified in detectable quantities. The key parameter that facilitated detection of in vitro activity and subsequent purification of DEBS1+TE and DEBS2 was the incubation temperature following IPTG (isopropylthio-β-D-galactoside) induction. Upon lowering the expression temperature from 30° C. to 22° C., active DEBS1+TE, DEBS2, and DEBS3 proteins could be detected in recombinant E. coli lysates. Hereafter, low temperature induction conditions were maintained throughout the course of this study.

The use of low temperatures in the favorable expression of large genes and proteins in E. coli suggests that other large genes and proteins can be expressed in E. coli as well as other organisms by beneficially using low temperatures as shown herein.

### EXAMPLE 3

Enhanced Production of 6-dEB in S. coelicolor

The presence of the matB and matC genes was also able to enhance the recombinant production of 6-dEB in S. coelicolor which had been recombinantly modified to produce this polyketide by insertion of the DEBS gene complex on the vector pCK7. The matB and matC genes were expressed in a recombinant strain of Streptomyces coelicolor that produces 50 mg/L 6-deoxyerythronolide B by virtue of plasmid borne DEBS genes. The matB and matC genes were inserted immediately downstream of DEBS genes on pCK7.

In more detail, the source of the matBC genes is pFL482, a derivative of PCR-Blunt (Invitrogen) containing a 5 kb BglII/HindIII fragment from pMATOP2 which carries the matBC genes. The NsiI fragment of pFL482 containing the matBC genes was cloned into the unique NsiI site of pCK7 in the same direction as the DEBS genes to yield pFL494. Upon transformation of plasmid pFL494 into S. coelicolor CH999, macrolide titer increases of 100-300% were obtained in the presence of exogenous methylmalonate (0.1-1 g/L).

Cultures of S. coelicolor CH999 with or without plasmid pCK7 or pFL494 were grown in flasks using R6 medium (sucrose, 103 g/L; K₂SO₄, 0.25 g/L; MgCl₂.6H₂O, 10.12 g/L; sodium propionate, 0.96 g/L; casamino acids (Difco), 0.1 g/L; trace elements solution, 2 mL/L; yeast extract (Fisher), 5 g/L; pH 7) supplemented with bis-tris propane buffer (28.2 g/L). Trace elements solution contained ZnCl₂, 40 mg/L; FeCl₃.6H₂O, 200 mg/L; CuCl₂.2H₂O, 10 mg/L; MnCl₂4H₂O, 10 mg/L; Na₂B₄O₇.1OH₂O, 10 mg/L; (NH₄)₆Mo₇0₂₄ .4H₂O. All media were supplemented with 50 mg/L thiostrepton (Calbiochem) to select for plasmid-containing cells, and with 5 mL/L Antifoam B (JT Baker) for control of foam. Thiostrepton was dissolved in DMSO prior to addition to cultures, giving a final DMSO concentration of approximately 1 mL/L of medium.

Seed cultures for the fermentation were prepared by inoculation of 50 mL medium, followed by growth for two days at 240 rpm and 30° C. in 250 mL baffled flasks (Bellco). These seed cultures were then used to inoculate 50 mL medium in the presence or absence of 1 g/L methylmalonate in 250-mL baffled flasks at 5% of final volume. All flask cultures were run in duplicate and sampled daily. The entire experiment was repeated once to ensure batch-to-batch reproducibility of the results.

Quantitation of 6-dEB and 8,8a-deoxyoleandolide was carried out using a Hewlett-Packard 1090 HPLC equipped with an Alltec 500 evaporative light scattering detector. HPLC samples were first centrifuged 5 min at 12,000×g to remove insolubles. The supernatant (20 μL) was applied onto a 4.6×10 mm column (Inertsil, C18 ODS3, 5 μm), washed with water (1 ml/min for 2 min), and finally eluted onto the main column (4.6×50 mm, same stationary phase and flow rate) with a 6-min gradient starting with 100% water and ending with 100% acetonitrile. 100% acetonitrile was then maintained for one min. Under these conditions, 6-dEB eluted at 6.2 minutes and 8.8a-deoxyoleandolide at 5.8 min. Standards were prepared from 6-dEB purified from fermentation broth. Quantitation error was estimated to be ±10%.

As described above, S. coelicolor CH999 either containing pCK7 or containing pFL494 were compared for their productivity of 6-dEB and 8,8a-deoxyoleandolide.

The results show the following:

1. Cell density was substantially the same for both strains.

2. The production of both 6-dEB and 8,8a-deoxyoleandolide is dramatically enhanced in CH999/pFL494 as compared to CH999/pCK7, whether measured in terms of mg/liters/hour or in mg/liter as a final titer after six days. (8,8a-deoxyoleandolide is the same as 6-dEB except that it contains methyl instead of ethyl as position 12, since acetyl CoA rather than propionyl CoA is used as a starter unit.) More specifically, after six days CH999/pFL494 plus methylmalonic acid produced 180 mg/l 6-dEB and about 90 mg/l of 8,8a-deoxyoleandolide. If methylmalonic acid was not added to the medium, 6-dEB was produced at a level of 130 mg/l while 8,8a-deoxyoleandolide was produced at bout 40 mg/l. For CH999 modified to contain pCK7, in the presence of methylmalonic acid in the medium, only 60 mg/l 6-dEB were formed along with about 20 mg/l of 8,8a-deoxyoleandolide. Without methylmalonic acid, these cells produced slightly less of each of these macrolides.

3. CH999/pFL494 completely consumed methylmalonate supplied at 1 g/L by day 6.

4. Consumption of 1 g/L methylmalonate results in a cumulative increase in macrolide of 200 m/L, representing a 35% conversion efficiency of methylmalonate into products.

5. CH999/pFL494 shows improved production of both macrolides even in the absence of exogenous methylmalonate (see 2 above).

6. Even CH999/pCK7 showed a 20% improvement in 6-dEB production when exogenous methylmalonate was added (see 2 above).

In addition to enhancing the productivity of known polyketides in natural and heterologous hosts, MatB is also used to produce novel polyketides. In contrast to other enzymes that produce the alpha-carboxylated CoA thioester building blocks for polyketide biosynthesis, such as methylmalonyl-CoA mutase (which has a high degree of specificity for succinyl-CoA) and acetyl/propionyl-CoA carboxylase (which prefers acetyl-CoA and/or propionyl-CoA), MatB is active with respect to a wide range of substrates. In addition to malonate and methylmalonate, MatB is able to activate substrates such as ethylmalonate, dimethylmalonate, isopropylmalonate, propylmalonate, allylmalonate, cyclopropylmalonate, and cyclobutylmalonate into their corresponding CoA thioesters.

Incorporation of these substrates into polyketide synthases requires a suitable acyltransferase (AT) which may be engineered into the appropriate module of a polyketide synthase, so that it can accept the unnatural substrate. Though none of these dicarboxylic acids yield detectable quantities of novel compounds when fed to CH999/pFL494, certain PKS enzymes naturally possess AT domains with orthogonal substrate specificity. For example, the FK506 PKS contains an acyltransferase domain that ordinarily incorporates bulky substrates such as propylmalonyl-CoA in preference to substrates such as malonyl-CoA or methylmalonyl-CoA, and can thus accept MatB-generated unnatural building blocks without any PKS engineering.

Using a protein engineering strategy described by Lau, J., et al.,, Biochemistry (1999) 38:1643-1651, the AT domain of module 6 of DEBS in pFL494 was modified to include the specificity determining segment from the niddamycin AT4 domain which incorporates ethylmalonyl-CoA. See: Kakavas, S. J., et al., J. Bacteriol (1997) 179:7515-7522. The resulting plasmid pFL508 was transformed into CH999. Upon feeding this strain with ethylmalonate, mass spectroscopy was able to detect a product corresponding to 2-ethyl-6dEB in levels comparable to that of 6dEB. The new compound was undetectable in the absence of ethylmalonate or in a control strain lacking the matBC genes.

### EXAMPLE 4

Production of 6-dEB in E. coli

We have demonstrated the ability of E. coli to produce complex, complete, polyketides, when programmed with the ability to express a functional PKS, a pantetheinyltransferase, and one or more pathways for producing starter and extender units. E. coli strain BL-21(DE) obtained from Novagen was modified genetically by inserting the phosphopantetheinyl transferase gene (the sfp gene) from Bacillus subtilis into the chromosome under the control of the phage T7 promoter by deleting the prpA-D portion of the prp operon, thus also placing the prpE locus, which encodes a propionyl CoA synthetase, under control of the T7 promoter. This genetically modified strain was then modified to include expression systems for the three genes encoding the DEBS1, DEBS2, and DEBS3 proteins, also under control of the T7 promoter as well as genes encoding propionyl CoA carboxylase and a gene encoding biotin ligase which is necessary for activation of the propionyl CoA carboxylase enzyme. The resulting E. coli contains a complete synthase for 6-dEB, a phosphopantetheinyl transferase necessary for the activation of this PKS, the propionyl CoA carboxylase enzymes that supply methylmalonyl CoA from propionyl CoA, and an inducible means to produce the endogenous propionyl CoA synthase capable of converting exogenous propionate to propionyl CoA. In addition, the endogenous system for catabolism of propionate was disarmed.

Thus, the E. coli are provided enzymes for synthesis of both starter and extender units under control of an inducible promoter, the endogenous mechanism for destruction of the propionate precursor of the starter and extender units has been disarmed; and expression systems (also under inducible promoters) have been provided for the necessary PKS proteins along with an expression system for the enzyme for activation of the PKS proteins.

In more detail, the genetically modified BL-21(DE3) strain was prepared according to the procedure described in Hamilton, et al., J. Bacteriol (1989) 171:4617-4622, which is incorporated herein by reference. A derivative of pMAK705 described in this publication, was prepared. In the derived vector, a T7 promoter coupled to the sfp gene was flanked by a 1,000 base pair sequence identical to that upstream of the A locus of the prp operon and a 1,000 base pair sequence identical to the sequence downstream of the E locus of this operon. The sfp gene was obtained from pUC8-sfp, a plasmid described by Nakano, et al., Mol. Gen. Genet. (1992) 232:313-321. The resulting integrated sequence deletes the prp loci A-D and inserts the T7 promoter controlling the sfp gene in their place and further results in placing the prpE locus under the control of the T7 promoter. As suggested herein, this site was chosen for sfp gene insertion for two reasons. First, the prp operon is putatively responsible for propionate catabolism in E. coli (Horswill, A. R., and Escalante-Semerena, J. C., J. BacterioL (1999) 181:5615-5623). Since propionate was intended to be the sole source of carbon building blocks for 6dEB biosynthesis (see below), concurrent propionate catabolism and anabolism were deemed undesirable. By deleting prpRBCD in the process of sfp integration, the ability of BAP1 to utilize propionate as a carbon and energy source was eliminated. Second, together with the sfp gene, the prpE gene in BAP 1 was also placed under control of an IPTG-inducible promoter such as a T7 promoter. PrpE is thought to convert propionate into propionyl-CoA (Horswill, A. R., and Escalante-Semerena, J. C., Microbiology (1999) 145:1381-1388); therefore, in the presence of exogenous propionate, propionyl-CoA can be expected to accumulate inside the cell at the same time as DEBS is expressed in an active form. It is noted, however, that it may not be desirable to delete prpRBCD is a production strain. It may be desirable in some strains, alternatively, to inactivate only some of the prpRBCD genes. The T7 promoter is inducible by IPTG.

The resulting genetically altered host, designated BAP 1, was than transfected with three plasmids each selectable for a different antibiotic resistance. These plasmids are as follows:

pBP130 is derived from pET21 (carb^{R}) obtained from Novagen and modified to contain the DEBS2 and DEBS3 genes under control of the T7 promoter.

pBP144 is a modified form of pET28 (kan^{R}) also available from Novagen containing the pcc and DEBS1 genes, also under control of the T7 promoter.

pCY214 (cm^{R}) contains the E. coli birA (biotin ligase) gene under the ara promoter and is described in Chapman-Smith, et al., Biochem. J. (1994) 302:881-887. This plasmid was obtained as a gift from Dr. Hugo Gramajo. The PCC protein and pcc gene are described in Rodriguez, et al., Microbiol. (1999) 145:3109-3119.

Construction of plasmids pBP130, pBP144: The expression vectors pET21c and pET28a were first re-engineered by replacing the Bpu1102I-DraIII fragments in these vectors with a polylinker possessing the Bpu1102I, NsiI, PstI, PacI and DraIII sites. The DEBS2 gene from pBP49 and the DEBS3 gene from pRSG50 were cloned into the pET21c derivative between the NdeI-EcoRI and NsiI-PacI sites, respectively, yielding pBP130 (25.5 kb). Thus, pBP130 is capable of expressing the DEBS2 and DEBS3 genes under the control of the same pT7 promoter. Similarly, pBP144 (20 kb) was constructed from the pET28a derivative described above by inserting the pccAB genes from pTR132 (Rodriguez, E., and Gramajo, H., Microbiology (1999) 145:3109-3119) and the DEBS1gene into the NdeI-EcoRI and PstI-PacI sites, respectively. This DEBS1 gene was derived from pRSG32 by replacing the SpeI-EcoRI fragment with a fragment amplified from the 3′ end of the natural DEBS1 gene using the following oligonucleotides: 5′ oligonucleotide: TTACTAGTGAGCTCGGCACCGAGGTCCGGGG; 3′ oligonucleotide: TTGAATTCGGATCGCCGTCGAGCTCCCGGCCGA. Thus, pBP144 expresses the pccAB genes and the DEBS1 gene, each under the control of its own pT7 promoter.

For the production of 6-dEB, BAP1 cells transformed with pBP130, pBP144, and pCY214 were grown in M9 minimal media with the appropriate antibiotics. The culture was grown to mid-log phase, followed by induction with IPTG and arabinose and the concomitant addition of 250 mg/L ¹³C-1-propionate. Induced cultures were grown for 12-24 hrs at 22° C. (Both the minimal medium and lower temperatures were found to be beneficial for DEBS gene expression. This protocol permitted growth-related production of 6-dEB, since glucose provided the carbon and energy source for general metabolism, while propionate was converted into 6-dEB.)

After 12-24 h the culture supernatant was extracted with ethyl acetate. The organic phase was dried in vacuo, and re-dissolved in CDCl₃ for ¹³C-NMR analysis. The accompanying spectrum showed that 6-dEB was the major ¹³C-labeled product. Other major ¹³C-labeled compound(s) with peaks in the range of 120-140 ppm are not derived from propionate incorporation, as confirmed by a separate experiment in which ¹³C-3-propionate was used in lieu of ¹³C-1-propionate. From the intensities of peaks corresponding to 6-dEB, it is estimated that at least 75% of the exogenous propionate was converted into 6-dEB. This was consistent with the disappearance of the propionate signal from the ¹³C NMR spectrum of the culture medium at the end of the fermentation. Negative control strains, which lacked either pBP130 or pBP144, failed to yield detectable quantities of 6-dEB.

The foregoing experiments were performed at low cell densities (OD₆₀₀ in the range of 0.5-2.5); a major advantage of synthesizing recombinant products in E. coli is that this bacterium can be grown to extremely high cell densities (OD₆₀₀ of 100-200) without significant loss in its specific catalytic activity.

The use of the matB and C genes or any of their homologs from other organisms in a non-native expression system is useful as a general strategy for the in vivo production of any alpha-carboxylated CoA thioester in any microbial host. The in vivo production of such CoA thioesters could be intended to enhance natural polyketide productivity or to produce novel polyketides. The matA gene is also useful to supplement in vivo levels of substrates such as acetyl-CoA and propionyl-CoA. Purified MatB is also used for the preparative in vitro production of polyketides, since CoA thioesters are the most expensive components in such cell-free synthesis systems.

### EXAMPLE 5

Incorporation of Diketides The BAP1 E. coli host organism described in Example 4 was transfected with p132 which contains an expression system for the PCCA and B subunits and with pRSG36 which contains an expression system for module 6+TE of DEBS3. The transfected cultures were grown on minimal selection media for both plasmids and then fed ¹⁴C labeled diketide. When induced and provided with propionate, ¹⁴C labeled triketide was obtained.

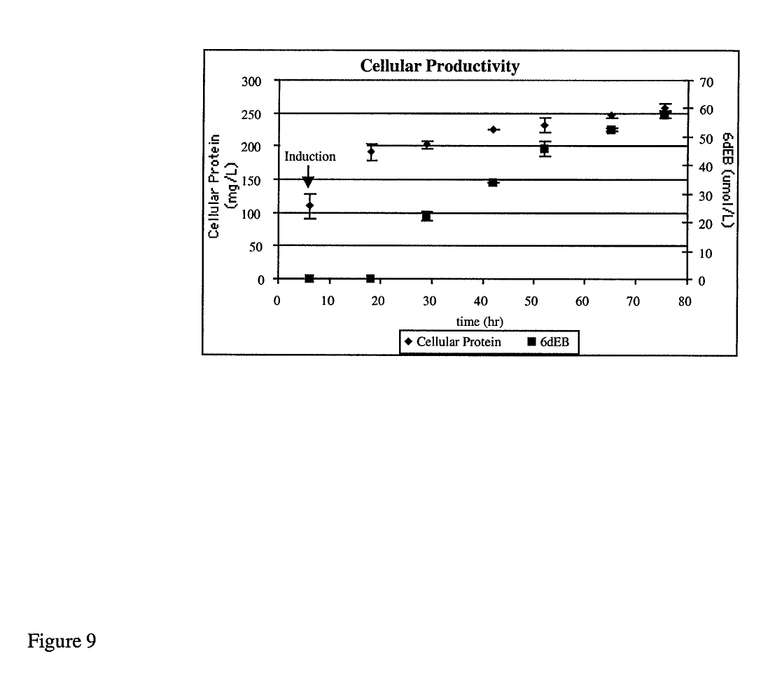

Alternatively, to co-express all three DEBS genes and the pcc genes, vectors pET21c and pET28a (Novagen) were modified to express two and three genes, respectively (The construction of plasmids pBP130 and pBP144 is described in Example 4.) When tested individually, protein production was observed from each gene located on both plasmids. BAP1 was transformed with these plasmids together with the birA plasmid. Individual transformants were cultured, induced and analyzed similar to the experiment for DEBS1+TE (above) using [1-¹³C]-propionate. NMR analysis of the crude organic extract revealed 6-dEB as the major propionate-derived metabolite of these recombinant cells. The product was later purified by HPLC and subjected to mass spectrometry yielding a major peak of the expected mass. Plasmids pBP130, pBP144, and pCY216 were transformed into BAP1 as previously described. Culture conditions were identical to those described for ¹³C-1-propionate fed at 250 mg/L described above in Example 2. Cultures were sampled regularly over 3 days. Samples were centrifuged and the supernatant (either 2 or 20 μL) loaded onto a Hewlett-Packard 1090 HPLC using an initial 4.6×10 mm column (Inertsil, C18 ODS3, 5 μm), washed with water (1 ml/min for 2 min), and then loaded onto a main 4.6×50 mm column with the same stationary phase and flow rate. A 6-minute gradient was then applied starting with 100% water and finishing with 100% acetonitrile maintained for an additional 1 minute. The samples were analyzed with an Alltech evaporative light scattering detection system (ELSD500), and a peak at 6.4 min retention time was confirmed as hepta-¹³C-labeled 6dEB by mass spectrometry (MW_{obs}=393). Product concentrations were measured in comparison to standard 6-dEB samples using the same detection scheme. At the end of the incubation period, the entire culture supernatant was extracted as before with ethyl acetate, dried, and analyzed by ¹³C-NMR. Additionally, the final cell pellet was analyzed via SDS-PAGE to confirm the presence of the three DEBS proteins and the PCC. No differences were observed between the expression levels of the proteins at 12 h and 48 h post-induction. The stability of each plasmid in BAP1/pBP130/pBP144/pCY216 was also tested at 12 h and 36 h post-induction. No loss of pBP144 was observed at either time-point, whereas pBP130 and pCY216 were maintained in 50% and 35% of the colonies at 12 h and 36 h, respectively. No rearrangement of any plasmid was detected at either time-point, based on restriction analysis of multiple re-transformed colonies. Negative controls for the ¹³C-NMR experiments included BAP1/pBP130/pCY216, BAP1/pBP144/pCY216, and BAP1/pBP130/pBP160/pCY216. (Plasmid pBP160 carries a C->. A null mutation at the active site of the KS domain in module 1 (Kao, C., et al., Biochemistry (1996) 35:12363). To quantify the productivity of this novel polyketide cellular system, culture samples were taken periodically, and the concentration of 6-dEB was measured (FIG. 9). From this data it can be calculated that the specific productivity of this cellular catalyst is 0.1 mmol 6dEB/g cellular protein/day. This is significantly superior to wild-type S. erythraea and compares well to an industrially relevant strain that overproduces erythromycin (0.2 mmol erythromycin/g cellular protein/day) (Minas, W., et al., Biotechnol Prog. (1998) 14:561) as a result of a decades-long program of directed strain improvement based on random mutagenesis.

### EXAMPLE 6

Construction, Expression, and Purification of the A-T Loading Didomain

The A-T loading didomain is naturally present at the N-terminus of RifA. To investigate this didomain biochemically, it was removed from the RifA protein context. Therefore, the sequence encoding the isolated A-T didomain was subcloned into an expression vector, using an NdeI restriction site engineered at the transcriptional start site of RifA and a NotI restriction site introduced in the linker region between the C-terminal end of the consensus T domain and the N-terminal end of the consensus ketosynthase domain of module 1, as described below in more detail. Thiolation domains require covalent attachment of the 4′-phosphopantetheine moiety of CoA to a conserved serine to be active (Walsh, C. T., et al. (1997) Curr. Opin. Chem. Biol. 1, 309-315). The Sfp phosphopantetheinyl transferase from B. subtilis, which is capable of converting the apo forms of many heterologous recombinant proteins into the holo forms, was therefore co-expressed with the A-T didomain in the holo enzyme preparation (Lambalot, R. H., et al. (1996) Chem. Biol. 3, 923-936; Quadri, L. E. N., et al. (1998) Biochemistry 37, 1585-1595). The apo and holo forms of the A-T didomain were produced in E. coli as C-terminal hexahistidine-tagged fusion proteins and were purified by nickel affinity chromatography to >98% homogeneity, as describe more fully below. Purified recombinant apo and holo A-T didomain (encoded by plasmid pSA8) were overproduced in E. coli, and protein samples were resolved by sodium dodecyl sulfate polyacrylamide gel electrophoresis (SDS-PAGE) (4-15%, Bio-Rad) and stained with SimplyBlue Safestain (Invitrogen). The apo A-T didomain and the holo A-T didomain each had a molecular weight of less than the 75 kD molecular weight marker. The holo A-T didomain had a slightly higher molecular weight that the apo A-T didomain.

Materials. [7-¹⁴C]-Benzoic acid (57 mCi/mmol) and [7-¹⁴C]-3-hydroxybenzoic acid (55 mCi/mmol) were obtained from American Radiolabeled Chemicals. All other substituted benzoic acids, phenylacetic acid, and 3-hydroxyphenylacetic acid were obtained from Aldrich in unlabeled form. ATP, CoA, and benzoyl-CoA were supplied by Sigma Chemical Company. AHB was synthesized according to a previously published protocol (Ghisalba, O., et al. J. Antibiot. (1981) 34:64-71). Restriction enzymes were from New England Biolabs.

Manipulation of DNA and Strains. DNA manipulations were performed in E. coli XL1 Blue (Stratagene) using standard culture conditions. Sambrook, J., et al. (1989) Molecular Cloning: A Laboratory Manual, 2nd ed., Cold Spring Harbor Laboratory Press, Plainview, N.Y. Polymerase chain reactions were carried out using Pfu polymerase (Stratagene) as recommended by the manufacturer.

Construction of an Expression Vector for the A-T Didomain. An NdeI restriction site was engineered at the start codon of the rifa gene using the primers 5′-GCGGCCATATGCGCACCGATCTC-3′ and 5′-AGGGCCCGCTGGCGGGAGAAC-3′ (mutated bases are shown in bold, and the introduced NdeI restriction site is underlined); the amplified 2.5 kb fragment was ligated to linearized pCR-Script (Stratagene) to produce pHu29. The rifA gene with the engineered NdeI restriction site at the start codon was then reconstructed in pHu90-1, a derivative of pRM5 (McDaniel, R., et al. (1993) Science 262, 1546-1550), via pHu29, pHu35, pHu50, and pHu51. Flanking restriction sites for PacI and PstI were used to transfer the sequence encoding the loading didomain and part of module 1 from pHu90-1 into a pUC18 derivative to produce pSA2. The loading didomain and module 1 are separated by an ˜20 amino acid linker region, delineated by the C-terminal end of the consensus T domain of the loading didomain and the N-terminal end of the consensus ketosynthase domain of module 1 (GenBank accession no. AF040570). To isolate the loading didomain from module 1, a NotI restriction site was introduced into the linker sequence using the primers 5′-ACCGAGACCTGCGGGGCGATCA-3′ and 5′-GCGGCCGCGACGGCCTGCGTG-3′ (mutated bases are shown in bold, and the introduced NotI restriction site is underlined); the resulting 0.94 kb fragment encodes from within the loading didomain into the linker region. This amplified fragment was ligated to linearized pCR-Blunt (Invitrogen) to produce pSA4, which was then digested with BamHI and PstI and ligated to pSA2 digested with the same enzymes to generate pSA6. The 1.9 kb NdeI-NotI fragment derived from pSA6 was ligated to NdeI-NotI-digested pET21c (Novagen) to produce pSA8, an expression vector for the loading didomain with hexahistidine appended to its C-terminus.

Expression and Purification of the A-T Didomain. Plasmid pSA8 was introduced via transformation into E. coli BL21 (Stratagene) for expression of the apo A-T didomain. One liter cultures of BL21/pSA8 were grown at 37° C. in 2 L flasks containing LB medium supplemented with 100 μg/mL carbenicillin. Expression of the A-T didomain was induced with 100 μM IPTG at an optical density at 600 nm of 0.7. After induction, incubation was continued for 6 h at 30° C. The cells were then harvested by centrifugation at 2500×g and resuspended in disruption buffer [200 mM sodium phosphate (pH 7.2), 200 mM sodium chloride, 2.5 mM DTT, 2.5 mM EDTA, 1.5 mM benzamidine, pepstatin (2 mg/L), leupeptin (2 mg/L), and 30% v/v glycerol].

All purification procedures were performed at 4° C. The resuspended cells were disrupted by two passages through a French press at 13,000 psi, and the lysate was collected by centrifugation at 40,000×g. Nucleic acids were precipitated with polyethylenimine (0.15%) and removed via centrifugation. The supernatant was made 45% (w/v) saturated with ammonium sulfate and precipitated overnight. After centrifugation, the pellet containing protein was redissolved in 50 mM tris(hydroxymethyl)aminomethane hydrochloride (Tris-HCl)(pH8), 300 mM sodium chloride, 10 mM imidazole, and 10% v/v glycerol. This solution was loaded onto a previously equilibrated nickel-nitrilotriacetic acid (Ni-NTA) column (2 mL, Qiagen). The column was washed with 20 mM imidazole in 50 mM Tris-HCl (pH 8), 300 mM sodium chloride, and 10% v/v glycerol, and the A-T didomain was eluted with 100 mM imidazole in the same solution. Pooled fractions containing the A-T didomain were buffer exchanged into 100 mM sodium phosphate (pH 7.2), 2.5 mM DTT, 2 mM EDTA, and 20% v/v glycerol by gel filtration (PD-10, Pharmacia) and concentrated with a Centriprep-50 concentrator (Amicon). The purified protein was flash-frozen in liquid nitrogen and stored at −80° C. Protein concentration was determined using the calculated extinction coefficient at 280 nm :49500 M⁻¹cm⁻¹ (Gill, S. C., et al. (1989) Anal. Biochem. 182, 319-326). A typical 1 L culture produced about 30 mg of purified protein.

For expression of the holo A-T didomain, plasmid pSA8 was transformed into BL21 containing the plasmid pRSG56 (Gokhale, R. S., et al. (1999) Science 284, 482-485), which carries a kanamycin resistance gene and the sfp gene. The sfp gene expresses Sfp, a non-specific phosphopantetheinyl transferase from B. subtilis that converts the apo protein into the holo protein (Lambalot, R. H., et al. (1996) Chem. Biol. 3, 923-936; Quadri, L. E. N., et al. (1998) Biochemistry 37, 1585-1595). One liter cultures of this recombinant E. coli strain were grown at 37° C. in 2 L flasks containing LB medium supplemented with 100 μg/mL carbenicillin and 50 μg/mL kanamycin. The expression and purification steps for the holo A-T didomain were performed as described above for the apo A-T didomain.

### EXAMPLE 7

Radioactive Labeling of A-T Didomain to Determine Mechanism of the A-T Didomain

For qualitatively assessing the incorporation of B or 3-HB into the A-T didomain, reactions contained 5 μM apo or holo A-T didomain, 50 mM sodium phosphate (pH7.2), 1 mM DTT, 1 mM EDTA, 15 mM MgCl₂, 10% glycerol, and 100 μM [7-¹⁴C]-B or [7-¹⁴C]-3-HB. In reactions where ATP was included, 5 mM was present. After incubation at 30° C. for 30 min, reactions were quenched with SDS-PAGE sample buffer and electrophoresed on a 4-15% gradient gel (Bio-Rad). The gel was briefly stained with Coomassie blue, destined, dried, and autoradiographed.

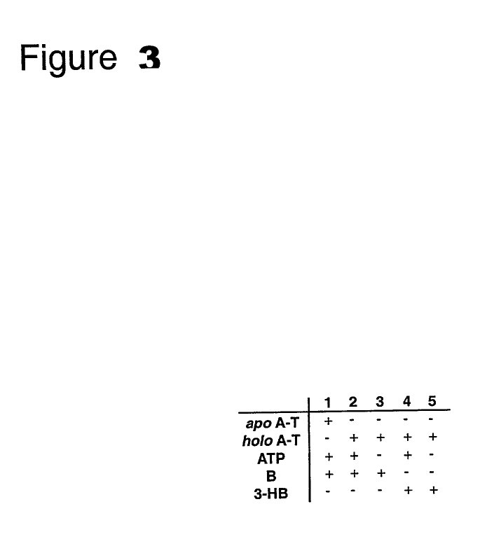

As depicted in FIG. 2, both models for the mechanism of the A-T didomain involve activation of AHB as the aryl-adenylate by the A domain, followed by eventual formation of a covalent aryl thioester enzyme intermediate from attack of either aryl-CoA (FIG. 2A) or the aryl-adenylate (FIG. 2B) by the thiol nucleophile of the phosphopantetheine cofactor of the T domain. To investigate these possible mechanisms, we sought to covalently load the A-T didomain. Although AHB is not available in radiolabeled form, in vivo feeding experiments have demonstrated that RifA can also be primed by 3-HB (Hunziker, D., et al. (1998) J. Am. Chem. Soc. 120, 1092-1093). Reactions containing [¹⁴C]-3- BB or the putative substrate [¹⁴C]-benzoate (B) and apo or holo A-T didomain were incubated in the presence or absence of Mg•ATP and subsequently analyzed by SDS-PAGE autoradiography (FIG. 3) as described in detail below. Lacking the phosphopantetheine cofactor, the apo A-T didomain could not be covalently loaded (lane 1). However, the holo A-T didomain is covalently loaded with both B and 3-HB in reactions that require Mg•ATP (lanes 2-5).

CoA was not included in the labeling reactions described above, suggesting that it is not required for covalent loading of the holo A-T didomain. Since the loading didomain has been proposed to be a CoA ligase (FIG. 2A) (Schupp, T., et al. (1998) FEMS Microbiol. Lett. 159, 201-207; August, P. R., et al. (1998) Chem. Biol. 5, 69-79; Ghisalba, O. et al. (1981) J. Antibiot. 34, 64-71), we nevertheless tested the possible involvement of CoA directly.

HPLC was used to detect the possible benzoyl-CoA formation according to the following procedure. Reactions contained 10 μM apo A-T didomain, 50 mM sodium phosphate (pH 7.2), 1 mM DTT, 1 mM EDTA, 15 mM MgCl₂, 5 mM ATP, 10% glycerol, 1 mM CoA, and 1 mM B. In reactions where benzoyl-CoA was included, 100 μM was present. After incubation at 30° C. for the indicated times, 20 μL samples were injected into an HPLC equipped with a C18 reverse phase column (VYDAC, 250×5 mm) with the detector monitoring at 254 nm. A linear gradient between buffer A (25 mM potassium phosphate, pH 5.4) and buffer B (100% acetonitrile) from 0% to 50% B was run over 14 min with a flow rate of 1 mL/min. The substrate and putative product peaks were identified by co-injection with authentic standards.

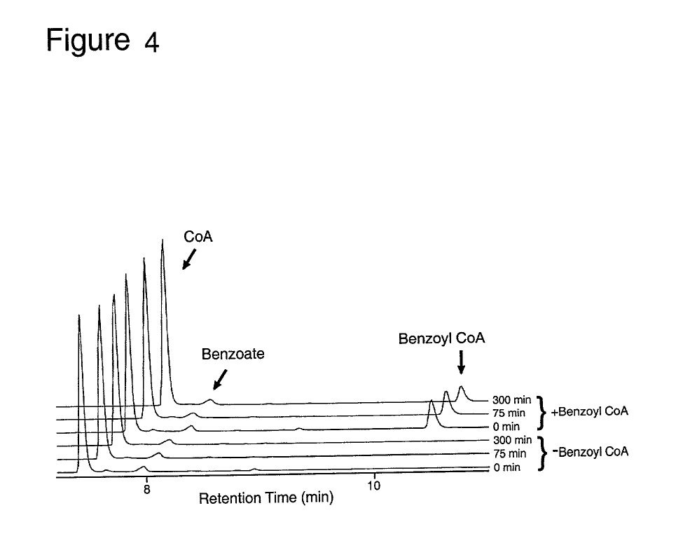

If the mechanism shown in FIG. 2A is operative, the apo A-T didomain should be capable of producing benzoyl-CoA. However, no benzoyl-CoA formation could be detected when the apo A-T didomain was incubated with ATP, B, and CoA (FIG. 4, -benzoyl-CoA traces). To confirm that benzoyl-CoA, if formed, would persist in these reaction conditions, benzoyl-CoA was added to an otherwise identical reaction (FIG. 4, +benzoyl-CoA traces). Benzoyl-CoA is degraded with an observed rate constant of ˜0.002 min⁻¹, and this degradation is enzyme-independent since the same observed rate constant is obtained for reactions in which the apo A-T didomain is omitted (data not shown); this slow nonenzymatic degradation is taken into account in the k_{cat} analysis that follows.

Accumulation of 5 μM benzoyl-CoA is readily detectable using this HPLC assay. This conservative detection limit allows an upper limit for k_{cat} for the formation of benzoyl-CoA by the apo A-T didomain to be calculated, as follows. Accumulation of 5 μM benzoyl-CoA would indicate that at most 10 μM benzoyl-CoA was formed during the 300 min reaction, as the half-life of benzoyl-CoA is ˜300 min under these conditions (t_{½}=1n2/k_{obs};·k_{obs}≈0.002 min⁻¹). Therefore, the velocity of benzoyl-CoA formation is at most 0.03 μM/min (10 μM/300 min). This corresponds to k_{cat}<0.003 min⁻¹, as the concentration of the apo A-T didomain in these reactions was 10 μM (k_{cat}=v/[E]ₜ). As described below, k_{cat} for covalent loading of the holo A-T didomain with B is 0.14 min⁻¹. Therefore, benzoyl-CoA is not a competent intermediate in the arylation reaction, as the rate constant for its formation is at least 50-fold less than the rate constant for formation of E-B. These results indicate that the CoA ligase model depicted in FIG. 2A is not viable for the A-T loading didomain of rifamycin synthetase.

### EXAMPLE 8

Direct Measurement of Kinetic Parameters for the Holo A-T Didomain

Typical reactions contained 1-10 μM holo A-T didomain, 50 mM sodium phosphate (pH 7.2), 1 mM DTT, 1 mM EDTA, 5 mM ATP, 15 mM MgCl₂, 10% glycerol, 0.5-5 μCi/mL [7-¹⁴C]-B or [7-¹⁴C]-3-HB, and varying concentrations of unlabeled B or 3-HB. Unlabeled B and 3-HB stocks were adjusted to the reaction pH prior to addition. Reactions were incubated at 30° C., and at desired time points 20 μL aliquots were quenched in 1 mL of ice-cold 5% trichloroacetic acid and 200 μg of bovine serum albumin (Sigma) was added to this mixture to aid precipitation of the protein. The precipitate was pelleted by centrifugation, washed with 0.5 mL of 5% trichloroacetic acid and solubilized in 0.5 mL of a 100 mM phosphate (pH 8), 2% SDS solution. This solution was combined with 4.5 mL of liquid scintillation fluid (Formula 989, Packard), and the incorporated ¹⁴C label, corresponding to E-B or E-3-HB, was quantified by liquid scintillation counting. Reaction rates were linearly dependent on enzyme concentration. Data analysis was performed using Kaleidagraph (Synergy Software), and exponential fits to the data typically gave R≧0.99.

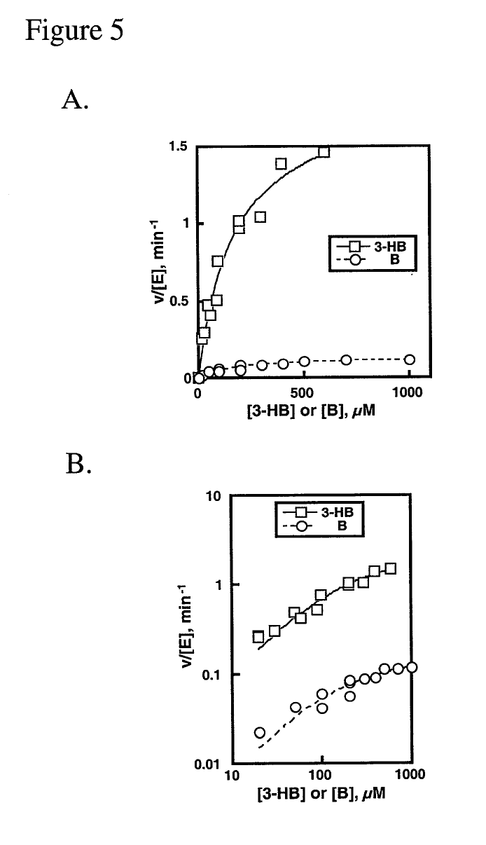

B and 3-HB are substrates for the holo A-T didomain, as shown qualitatively in FIG. 3. To quantitatively assess these benzoates as substrates for aryl-adenylate formation followed by arylation of the thiol of the phosphopantetheine cofactor of the T domain, we utilized the protein precipitation assay described above. As discussed above, aliquots from reactions containing holo A-T didomain, 0.5-5 μCi/mL [7-¹⁴C]-B or [7-¹⁴C]-3-HB, and varying concentrations of unlabeled B or 3-HB were quenched with trichloroacetic acid, and the amount of radiolabeled protein in each washed protein pellet was determined by liquid scintillation counting. Initial velocities of E-B or E-3-HB formation as a function of B or 3-HB concentrations were obtained using this method and used to generate the saturation curves shown in FIG. 5. Best fits of the data to a saturation model give a k_{cat} of 1.9 min⁻¹ and K_{M} of 180 μM for 3-HB, and a k_{cat} of 0.14 min⁻¹ and K_{M} of 170 μM for B. The ratio of k_{cat}/K_{M} values for the two substrates reveals a 12-fold preference for 3-HB over B by the A-T didomain. Addition of CoA to these reactions had no effect (data not shown), consistent with the conclusion that the A-T didomain is not a CoA ligase.

### EXAMPLE 9

Chase Experiment to Screen for Substrate Specificity of the A-T Didomain

Reactions were carried out in 50 mM sodium phosphate (pH 7.2), 1 mM DTT, 1 mM EDTA, 5 mM ATP, 15 mM MgCl₂, and 10% glycerol. Each reaction additionally contained 20 μM holo A-T didomain and 0.5 mM of a putative substrate, 0.5 mM unlabeled B, or no added substrate. After incubation for 30 min at 30° C., 100 μL reaction aliquots were applied to individual G-25 microspin gel filtration columns (Pharmacia) that had been pre-equilibrated with the reaction buffer. The protein component of the applied sample was eluted from the microspin column in constant volume by centrifugation, according to the manufacturer's instructions. A 10 μL aliquot of each eluted protein sample was diluted with 2 μL of a [7-¹⁴C]-B solution, for a final B concentration of 200 μM. These chase reactions were incubated for 15 min at 30° C. prior to analysis by SDS-PAGE autoradiography.

Based on previous in vivo feeding experiments (Hunziker, D., et al., J. Am. Chem. Soc. (1998) 120:1092-1093) and the in vitro results just described, AHB, 3-HB, B, and 3,5-dihydroxybenzoate are accepted as substrates by the A-T didomain.

To screen for additional substrates that can prime the A-T didomain, the simple chase experiment was devised as described above. Holo A-T didomain was first incubated with a putative substrate under standard reaction conditions. The reaction mixture was then passed over a microspin gel filtration column to separate the protein components from the putative unreacted substrate. Radiolabeled B was finally added to the protein fraction, and the mixture was incubated briefly prior to SDS-PAGE autoradiography. Protein samples that had originally been incubated with a substrate would contain covalently loaded enzyme-substituted benzoate (E-XB), which would not react with radiolabeled B during the chase, resulting in little or no detectable enzyme-benzoate (E-B) by SDS-PAGE autoradiography. In contrast, protein samples that had originally been incubated with a poor substrate or a non-substrate would primarily contain free enzyme (E), which would readily react with radiolabeled benzoate (B) during the chase to form E-B, resulting in a radioactive band detectable by SDS-PAGE autoradiography.

The results of this screening experiment for a series of substituted benzoates are discussed below. An autoradiograph of a gel (4-15%, Bio-Rad) containing A-T didomain samples chased with radiolabeled B after incubation with no substrate; unlabeled B; 2-aminobenzoate; 3-aminobenzoate; 4-aminobenzoate; AHB; 3-amino-4-hydroxybenzoate; 4-amino-2-hydroxybenzoate; 3-bromobenzoate; 3-chlorobenzoate; 3,5-diaminobenzoate; 3,5-dibromobenzoate; 3,5-dichlorobenzoate; 3,5-difluorobenzoate; 2,3-dihydroxybenzoate; 3,5-dihydroxybenzoate; 3,5-dinitrobenzoate; 3-fluorobenzoate; 2-hydroxybenzoate; 3-HB; 4-hydroxybenzoate; 3-methoxybenzoate; 3-nitrobenzoate; 3-sulfobenzoate.

The first two lanes contain control reactions in which no substrate (lane 1) or unlabeled B (lane 2) was present in the initial incubation; as expected, radiolabeled A-T didomain was formed in the no substrate control reaction but not in the unlabeled B control reaction. Radiolabeled A-T didomain is likewise absent from reactions in which the known substrates AHB (lane 6), 3,5-dihydroxybenzoate (lane 16), and 3-HB (lane 20) were present in the initial incubation. In addition to these three substrates, ten more likely substrates were identified for further investigation based on the absence or diminution of radiolabeled A-T didomain as compared to the lane 1 control reaction. These ten substrates are 2-aminobenzoate; 3-aminobenzoate; 3-bromobenzoate; 3-chlorobenzoate; 3,5-diaminobenzoate; 3,5-dibromobenzoate; 3,5-dichlorobenzoate; 3,5-difluorobenzoate; 3-fluorobenzoate; and 3-methoxybenzoate. Although the simplest model for the absence of radiolabeled A-T didomain in a given reaction is that the substituted benzoate in question has been loaded onto the A-T didomain, blocking the enzyme from reaction with radiolabeled B during the chase, this experiment does not rule out the possibility that it is instead a tight binding competitive inhibitor. However, the observation described below that the competition between these substituted benzoates and the substrate B is time-independent renders the inhibition model unlikely. Radiolabeled A-T didomain was formed in the reactions having the following substrates: 4-aminobenzoate; 3-amino-4-hydroxybenzoate; 4-amino-2-hydroxybenzoate; 2,3-dihydroxybenzoate; 3,5-dinitrobenzoate; 2-hydroxybenzoate; 4-hydroxybenzoate; 3-nitrobenzoate; and 3-sulfobenzoate.

### EXAMPLE 10

Relative Specificity Determination Using Relative Rate Constants for Arylation of the A-T Didomain

Armed with the set of likely substrates found in the screening described in the chase experiment (Example 9), the relative specificity of the A-T didomain for aryl-adenylate formation followed by arylation of the thiol of the phosphopantetheine cofactor of the T domain was determined. Addition of a substituted benzoate to a reaction mixture containing radiolabeled benzoate (B) and the holo A-T didomain allowed partitioning between reaction with the substituted benzoate (XB) and reaction with B to be followed. Reactions were performed as described above (Example 8 Kinetic Measurements) but in the presence of 50 μM-5 mM of a series of substituted benzoates. Substituted benzoate stocks were adjusted to the reaction pH prior to addition. The rate constant relative to an analogous reaction with benzoate (kᵣₑₗ) for reaction of a given substituted benzoate with respect to reaction of B was determined from the concentrations of B and substituted benzoate in the original reaction ([B], [XB]) and the amount of product present as E-B and E-XB, according to the equation in Scheme 1 below. (Fersht, A. R. (1998) in Structure and Mechanism in Protein Science pp. 116-117, W. H. Freeman, New York.)

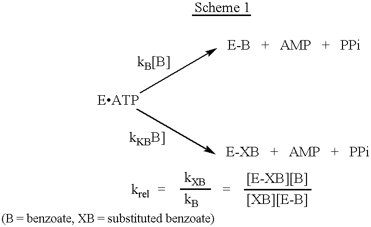

The amount of E-XB product in each reaction at a given time point was determined by subtracting the amount of radiolabeled E-B in the presence of the competing substituted benzoate from that obtained at the same time point in an identical reaction lacking competitor. The ratio of E-B to E-XB was constant throughout a particular time course, indicating that no secondary reactions involving the reaction products were occurring. The constant ratios also support the view that the substituted benzoates are true substrates and not high affinity competitive inhibitors, as E-B would continue to accumulate in the presence of a competitive inhibitor, resulting in a ratio of E-B to apparent E-XB that increases as a function of time. For each substituted benzoate, the same kᵣₑₗ value, within error, was obtained for reactions performed at different substituted benzoate concentrations. The reactions were repeated for selected substituted benzoates using radiolabeled 3-HB instead of B, and the same kᵣₑₗ values (with respect to B), within error, were obtained. Each kᵣₑₗ value in Table 1 represents an average of at least 4 separate determinations. Competition with B for reaction with the A-T didomain by phenylacetate and 3-hydroxyphenylacetate could not be detected, so limits for kᵣₑₗ for these compounds are reported in Table 1.

The kᵣₑₗ values in Table 1 represent the k_{cat}/K_{M} ratio for a given substituted benzoate and B, and as such provide a measure of the specificity of the A-T didomain for each substrate (Fersht, A. R. (1998) in Structure and Mechanism in Protein Science pp. 116-117, W. H. Freeman, New York). The validity of this approach is demonstrated by comparing the kᵣₑₗ value of 12 obtained for 3-HB with the identical k_{cat}/K_{M} ratio of 12 obtained from direct measurement of k_{cat}/K_{M} for 3-HB and B (FIG. 4). The A-T didomain exhibits a 10-1000-fold preference for AHB, its biological substrate, over all other substrates.

### EXAMPLE 11

Construction of Plasmid pBP165

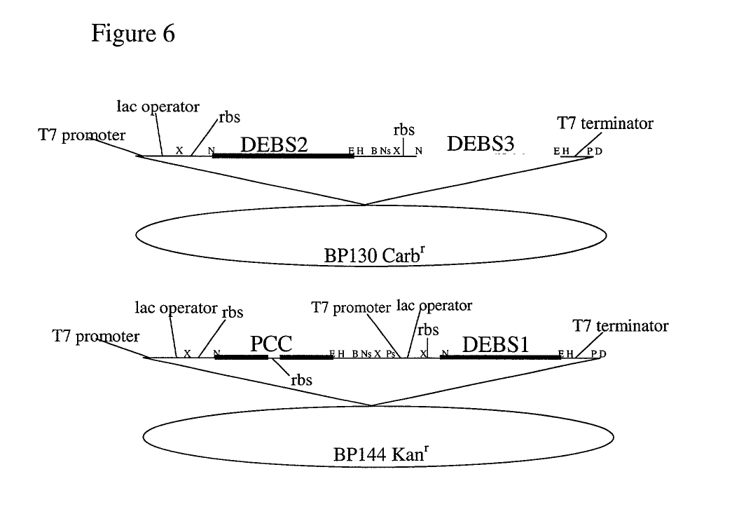

To engineer a functional fusion between the A-T loading didomain from the rifamycin synthetase and the first module of DEBS, the DNA sequence immediately upstream of the KS domain in DEBS module 1 was modified to read as follows: CCGGCGAACCGATCGCGATCGTCGCGATGG. The engineered BsaBI site (in bold) was fused to the corresponding naturally occurring BsaBI site between the A-T loading didomain and the first PKS module of the rifamycin synthetase (FIG. 6). The resulting fusion was transferred into pBP144 in place of DEBS1, giving rise to pBP165.

TABLE 1
Relative Rate Constants for Covalent Loading of the
A-T Didomain by Substituted Benzoates^{a}

| Substrate | kᵣₑₗ^{b} |
|---|---|
| 3-amino-5-hydroxybenzoate | 120 ± 10 |
| 3,5-diaminobenzoate | 16 ± 1 |
| 3-hydroxybenzoate | 12 ± 2 |
| 3-aminobenzoate | 6.6 ± 0.6 |
| 3,5-dibromobenzoate | 4.1 ± 0.5 |
| 3,5-dichlorobenzoate | 4.0 ± 0.5 |
| 3,5-dihydroxybenzoate | 3.1 ± 0.5 |
| 3-chlorobenzoate | 2.1 ± 0.2 |
| 3-bromobenzoate | 1.9 ± 0.2 |
| benzoate | (1) |
| 2-aminobenzoate | 0.62 ± 0.08 |
| 3-methoxybenzoate | 0.43 ± 0.06 |
| 3-fluorobenzoate | 0.42 ± 0.11 |
| 3,5-difluorobenzoate | 0.13 ± 0.02 |
| phenylacetate | <0.01 |
| 3-hydroxyphenylacetate | <0.01 |

^{a}30° C., 50 mM sodium phosphate, pH 7.2, 1 mM DTT, 1 mM EDTA, 15 mM MgCl₂, 5 mM ATP, 10% glycerol.
^{b}Rate constant for T domain arylation, relative to T domain arylation by benzoate

1. Procaryotic host cells which are genetically modified for enhanced synthesis of at least one polyketide, wherein said modification comprises incorporation of at least one expression system for producing a protein that catalyzes the production of starter and/or extender units and/or disabling at least one endogenous pathway for catabolism of starter and/or extender units.

2. A method to produce a polyketide which method comprises culturing the cells of claim 1 under conditions wherein said polyketide is produced.

3. A method to assess the results of a procedure effecting modification of polyketide synthase genes, resulting in a mixture of said modified genes which method comprises
transfecting a culture of cells of claim 1 with said mixture of modified genes, wherein said cells are E. coli,
culturing individual colonies of said transformed E. coli, and
assessing each colony for polyketide production

4. A method to determine whether a substituted benzoate can prime an adenylation-thiolation (A-T) didomain of a rifamycin synthetase comprising
incubating a substituted benzoate with a holo A-T didomain under conditions suitable for priming the A-T didomain; and
measuring the amount or presence of the substituted benzoate that primed the A-T didomain.

5. Procaryotic host cells which do not produce a polyketide in the absence of genetic modification and which are genetically modified for enhanced synthesis of at least one hybrid polyketide, wherein said modification comprises incorporation of at least one expression system comprising an A-T didomain, which incorporates a starter unit that primes an A-T didomain according to the method of claim 4.

6. The procaryotic host cells defined in claim 5 wherein the starter unit is selected from the group consisting of 2-aminobenzoate, 3-aminobenzoate, 4-aminobenzoate, 3-amino-5-hydroxybenzoate, 3-amino-4-hydroxybenzoate, 4-amino-2-hydroxybenzoate, 3-bromobenzoate, 3-chlorobenzoate, 3,5-diaminobenzoate, 3,5-dibromobenzoate, 3,5-dichlorobenzoate, 3,5-difluorobenzoate, 2,3-dihydroxybenzoate, 3,5-dihydroxybenzoate, 3,5-dinitrobenzoate, 3-fluorobenzoate, 2-hydroxybenzoate, 3-hydroxybenzoate, 4-hydroxybenzoate, 3-methoxybenzoate, 3-nitrobenzoate, and 3-sulfobenzoate to make a modified polyketide.

7. The procaryotic host cells defined in claim 5 wherein the starter unit is selected from the group consisting of 2-aminobenzoate, 3-aminobenzoate, 4-aminobenzoate, 3-amino-4-hydroxybenzoate, 4-amino-2-hydroxybenzoate, 3-bromobenzoate, 3-chlorobenzoate, 3,5-diaminobenzoate, 3,5-dibromobenzoate, 3,5-dichlorobenzoate, 3,5-difluorobenzoate, 2,3-dihydroxybenzoate, 3,5-dinitrobenzoate, 3-fluorobenzoate, 2-hydroxybenzoate, 4-hydroxybenzoate, 3-methoxybenzoate, 3-nitrobenzoate, and 3-sulfobenzoate to make a modified polyketide.

8. A hybrid polyketide in which a starter unit is incorporated therein which starter unit primes an A-T didomain according to the method of claim 4.

9. The hybrid polyketide defined in claim 8 where the starter unit is selected from the group consisting of 2-aminobenzoate, 3-aminobenzoate, 4-aminobenzoate, 3-amino-5-hydroxybenzoate, 3-amino-4-hydroxybenzoate, 4-amino-2-hydroxybenzoate, 3-bromobenzoate, 3-chlorobenzoate, 3,5-diaminobenzoate, 3,5-dibromobenzoate, 3,5-dichlorobenzoate, 3,5-difluorobenzoate, 2,3-dihydroxybenzoate, 3,5-dihydroxybenzoate, 3,5-dinitrobenzoate, 3-fluorobenzoate, 2-hydroxybenzoate, 3-hydroxybenzoate, 4-hydroxybenzoate, 3-methoxybenzoate, 3-nitrobenzoate, and 3-sulfobenzoate.

10. The hybrid polyketide defined in claim 9 where the starter unit is selected from the group consisting of 2-aminobenzoate, 3-aminobenzoate, 4-aminobenzoate, 3-amino-4-hydroxybenzoate, 4-amino-2-hydroxybenzoate, 3-bromobenzoate, 3-chlorobenzoate, 3,5-diaminobenzoate, 3,5-dibromobenzoate, 3,5-dichlorobenzoate, 3,5-difluorobenzoate, 2,3-dihydroxybenzoate, 3,5-dinitrobenzoate, 3-fluorobenzoate, 2-hydroxybenzoate, 4-hydroxybenzoate, 3-methoxybenzoate, 3-nitrobenzoate, and 3-sulfobenzoate.

11. A method to produce a polyketide which method comprises culturing the cells of claim 5 under conditions wherein said polyketide is produced.

12. The cells of claim 5 which are of the genus Escherichia, Streptomyces, Bacillus, Pseudomonas, or Flavobacterium.

13. The cells of claim 12 which are E. coli.

14. The cells of claim 5 wherein said cells produce a complete polyketide derived from rifamycin, rapamycin, FK506, ansatrienin, FK520, microcystin, pimaricin, erythromycin, oleandomycin, megalomycin, picromycin, spinosad, avermectin, tylosin or epothilone.

15. The cells of claim 14 which produce a modified rifamycin.

16. The cells of claim 14 which produce a 6-dEB analog.

17. The cells of claim 5, wherein said genetic modification further comprises incorporation of at least one expression system for a polyketide synthase protein.

18. The cells of claim 5 wherein said genetic modification comprises incorporation of at least one expression system for a phosphopantetheinyl transferase.

19. A method to enhance the production of at least one hybrid polyketide in a microbial host which method comprises providing said host with an expression system for producing a protein that incorporates an exogenous starter unit that primes an A-T didomain according to the method of claim 4.
